(19) United States
(12) Patent Application Publication
(10) Pub. No.: US 2007/0003115 A1
(43) Pub. Date: Jan. 4, 2007

(54) Remote diagnostic device for medical testing

(75) Inventors: David L. Patton, Webster, NY (US); Steven C. Switalski, Rochester, NY (US); Douglas B. Beaudet, Geneseo, NY (US); Richard W. Wien, Pittsford, NY (US); Nelson A. Blish, Rochester, NY (US)

Correspondence Address: Mark G. Bocchetti;Patent Legal Staff, Eastman Kodak Company, 343 State Street, Rochester, NY, 14650-2201, US

(73) Assignee: Eastman Kodak Company

(21) Appl. No.: 11/171,671

(22) Filed: Jun. 30, 2005

(51) Int. Cl. G06K 9/00; A61B 5/08; A61B 10/00

(52) U.S. Cl. 382/128; 600/572; 600/543

(57) ABSTRACT

A remote diagnostic device for medical testing includes a kit dispenser (315), a sampling device (85) contained in the kit (59), a receiving web (62) for receiving a sample (64) after use by a user (305), a processor for developing the latent image, a scanner (195) for detecting a developed image, and a microprocessor (205) for analyzing the scanned image for pathogens. The receiving web forms a latent image of any pathogens present in the sample.

### FIELD OF THE INVENTION

This invention relates to a remote diagnostic device such as a kiosk which uses a test kit incorporating a test strip or web for detecting bacteria, viruses, and other pathogens in humans and more specifically to diseases and a method for processing and viewing the results to make a preliminary diagnosis. The test strip or web uses silver halide amplification technology.

### BACKGROUND OF THE INVENTION

Easy and effective methods for remotely diagnosing diseases have long been sought. Antibody technology comprises the largest group of rapid methods; a large number of immunology-based rapid assays have been successfully used for detection of bacteria, toxins, cells, and viruses. Many forms of immunology-based rapid assays have been investigated and developed, including immunofiltration (IMF), micro array immunoassay (MAI), enzyme-linked immunofiltration (ELIFA), chemiluminescent immunoassay (CLIA), immunomagnetic separation (IMS), immunoliposome sandwich assay (ILSA), immunochromatography, and improved and standard applications of sandwich ELISA. Many of the above are commercially available, evaluated and validated under stringent requirement testing programs. Some rapid test systems incorporate more than one immunology-based technology into the test system to improve specificity and/or sensitivity, such as the use of IMF and ELISA or IMS and ELISA. Immunology-based rapid assays already in existence can be further modified or incorporated into other systems to improve their performance, which obviates the need to create entirely new detection systems.

Many rapid immunological test methods have been reported to deliver results within as little time as 10 minutes to as much as several hours. However, such methods must be used within the context of a total test system, which usually requires one or more additional, lengthier preparatory steps (8 to 24 hours) to selectively amplify the target prior to rapid testing. Thus, the term “rapid” does not necessarily apply to the entire test process, which in total can require more than a day to complete. While most rapid immunological methods have achieved ultimate detection steps of minutes, they still rely on pre-enrichment, immunocapture and/or preincubation steps in order to enhance inherent assay sensitivity and/or specificity.

Enzyme-based systems currently in commercial use for immunodetection lack the ability to adequately amplify the detection signal. The average working detection limit for these assays is on average 10³-10⁵ cells per ml or per gram of test material, achieved only after selective pre-enrichment and/or purification and concentration step is performed to reduce microbial background and to amplify the target organism. Without an additional amplification step, many of these tests would lack sufficient sensitivity to be useful.

An alternate approach to increasing sensitivity is to amplify the target signal detected within the immunodection system; some newer approaches have taken such an approach. Such a system must be robust, safe, portable, and usable by personnel with minimal laboratory training. Further, the test should be flexible enough to be adapted to possible new diseases and prevent cross contamination.

Patient medical records, and electronic representations of these records can be stored in multiple embodiments such as on a medical card, a computerized database system, or even dog tags on soldiers. U.S. Patent Application Publication No. 2004/0204961 (Rensimer et al.) discusses a system and method for processing patient data that permits physicians and other medical staff personnel to record historical patient care information. This patent publication teaches that the medical care data can be recorded, saved, and transferred from a portable system to a larger stationary information or database system. U.S. Patent Application Publication No. 2003/0177033 (Park et al.) teaches a method of transmitting an electronic patient record between doctors and pharmacies for prescription and treatment information, using the Internet.

Non-medical kiosks such as automated teller machines (ATMs) require user authentication in order to access personal records prior to performing a transaction. This authentication can take the form of submitting a bankcard and pin number, a user id and password, or more sophisticated biometric analysis. Unassisted medical kiosks exist in the marketplace and provide basic vital statistics monitoring such as patient heart rate and blood pressure (see LifeClinic at www.LifeClinic.com). U.S. Patent Application Publication No. 2004/0044560 (Giglio et al.) discusses a device to test and output the personal data (fat analysis) of a user to a computer processor. U.S. Pat. No. 6,692,436 (Bluth et al.) teaches a health kiosk that provides blood pressure testing, a health and fitness evaluation, and a medication encyclopedia. Other unassisted kiosks aid a user in diagnosing a condition by using question and answer scripts to reach a diagnostic conclusion. U.S. Pat. No. 6,641,532 (Iliff) teaches the art of conducting an automated diagnostic session with a patient, using a plurality of disease scripts, a patient medical record, and a disease engine to process the script and route the changes to the medical record. Staffed medical kiosks also exist that provide a nurse to check on certain ailments (see MinuteClinic at www.MinuteClinic.com).

All of these medical kiosks provide convenient medical services to consumers with improved accessibility over visits to a doctor's office. However, the unassisted kiosks are limited in their ability to provide comprehensive diagnostic services due to the lack of secure access to patient medical records (including doctor's orders, prescription information, and individual patient history) and the inability to perform diagnostic tests beyond basic vital statistic analysis or question and answer scripts. Although assisted kiosks can provide more diagnostic tests for patients, they are limited in convenience by their hours of operation, limited number of locations, and limited access to electronic patient records.

A need exists in the marketplace to further extend the utility of medical kiosks to provide a greater variety of tests in convenient, accessible locations; while ensuring that patient privacy, and the security and integrity of electronic medical records are maintained. There also exists the need for a patient when receiving a positive diagnosis to make an appointment with a specialist or doctor in a convenient, efficient and timely manner.

### SUMMARY OF THE INVENTION

Briefly, according to one aspect of the present invention a remote diagnostic device for medical testing includes a kit dispenser, a sampling device contained in the kit, a receiving web for receiving a sample after use by a user, a processor for developing the latent image, a scanner for detecting a developed image, and a microprocessor for analyzing the scanned image for pathogens. The receiving web forms a latent image of any pathogens present in the sample.

### BRIEF DESCRIPTION OF THE DRAWINGS

Other objects, advantages and features of the present invention will become apparent from the following specification when taken in conjunction with the drawings in which like elements are commonly enumerated and in which:

FIG. 1 illustrates a cross section of a structure of a typical multilayer sensor made in accordance with the present invention;

FIG. 2 illustrates a cross section of another embodiment of the multilayer sensor of FIG. 1;

FIG. 3 illustrates a cross section of yet another embodiment of the multilayer sensor of FIG. 1 made in accordance with the present invention;

FIG. 4A illustrates an embodiment of the multilayer sensor of FIG. 1 in the form of a test strip;

FIG. 4B illustrates an embodiment of the multilayer sensor of FIG. 1 in the form of a test web;

FIG. 4C is a schematic of the multilayer sensors of FIGS. 4A and 4B showing a sample present in the sampling area;

FIG. 4D illustrates yet another embodiment in the form of a test strip array comprising more than one of the test strips of FIG. 4A in accordance with the present invention;

FIGS. 5 and 6 are a schematics illustrating how a sample is obtained from human skin using the test strip of FIG. 4A;

FIGS. 7A and 7B are schematics illustrating another embodiment of how a sample is obtained from a test area such as the inside of the mouth or throat and applied to the test strip of FIG. 4A or the web of FIG. 4B;

FIG. 8 is a schematic of another embodiment of the swab shown in FIGS. 7A and 7B;

FIGS. 9 and 10 are schematics illustrating how the multilayer sensor of FIG. 1 can be heat processed;

FIG. 11A is a schematic of an apparatus used to process the latent image formed on the test strip and to scan the visual image after processing;

FIG. 11B is a schematic of another embodiment of the apparatus of FIG. 11A;

FIG. 12 is a schematic illustrating yet another embodiment of the present invention having multiple coatings for testing for multiple pathogens on the test strip of FIG. 4A or the test web FIG. 4B;

FIGS. 13 and 14 illustrate the multilayer sensor after it has been processed using the methods of FIG. 10 or 11 in accordance with the present invention;

FIGS. 15 and 16 illustrate another embodiment of the test strip after it has been processed using the methods of FIG. 10 or 11 in accordance with the present invention;

FIG. 17A illustrates a kiosk made in accordance with the present invention;

FIG. 17B illustrates the kiosk of FIG. 17A networked with other servers containing medical information and records;

FIG. 17C illustrates a flow chart for making an appointment using the kiosk of FIG. 17A made in accordance with the present invention;

FIGS. 18, 19 and 20 are schematic drawings of an apparatus used to apply a sample on a swab to a test strip in a kiosk;

FIG. 21 is a schematic drawing of the apparatus of FIGS. 18, 19, and 20 illustrating the disposal of a used swab in a kiosk;

FIG. 22 is a schematic drawing of another embodiment of an apparatus used for applying a sample to a test web in a kiosk;

FIG. 23 is an enlarged partial top view of the sampling area taken along line 23-23 of FIG. 22;

FIG. 24 is an enlarged cross section view of the sampling area taken along line 24-24 of FIG. 23 illustrating a user applying a sample to a test web in the kiosk;

FIG. 25 is a schematic of a device used to take a breath sample for testing in a kiosk; and

FIG. 26 is a schematic of an apparatus used to process the breath sample in a kiosk taken using the device in FIG. 25.

### DETAILED DESCRIPTION OF THE INVENTION

The test strip, test web, and test web array of the invention utilize the following described sensor technology. The sensor used in the invention takes advantage of the amplification properties of photographic silver halide. When a silver halide grain has as little as three constituent atoms reduced to silver (known as a “latent image”), the grain can be developed or completely converted to a grain of silver. The development may be done with chemical development (either time-released, triggerable, or manually with a development solution), or with heat development (as in dry film development systems, such as the Kodak DryView X-ray film system). The atoms changed to silver are usually triggered by light, and as little as three photons are needed to create the silver atom of the cluster forming the latent image. This means that a very small stimulus can be stored, and then amplified chemically by the silver halide grain itself, by more than a million fold.

In this sensor system the latent image is formed by the diffusion of chemically active species (signal compound) (e.g., free radicals, redox species, etc.) that are produced or released in the upper layers. Since these active chemical species are released by the interaction of the suspect pathogen with the upper layers of the film, the latent image is tied to the presence of the pathogen. Development of this latent image can either proceed spontaneously, as the latent image builds up from the original dose, or can be triggered chemically or thermally. The triggered development has all the amplification capability of the silver halide grain.

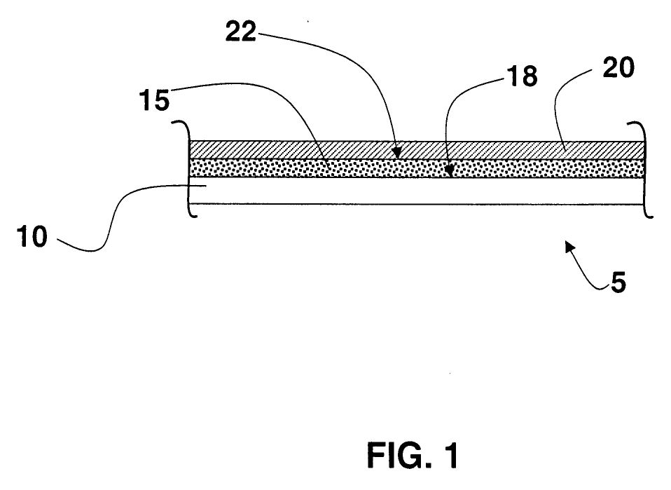

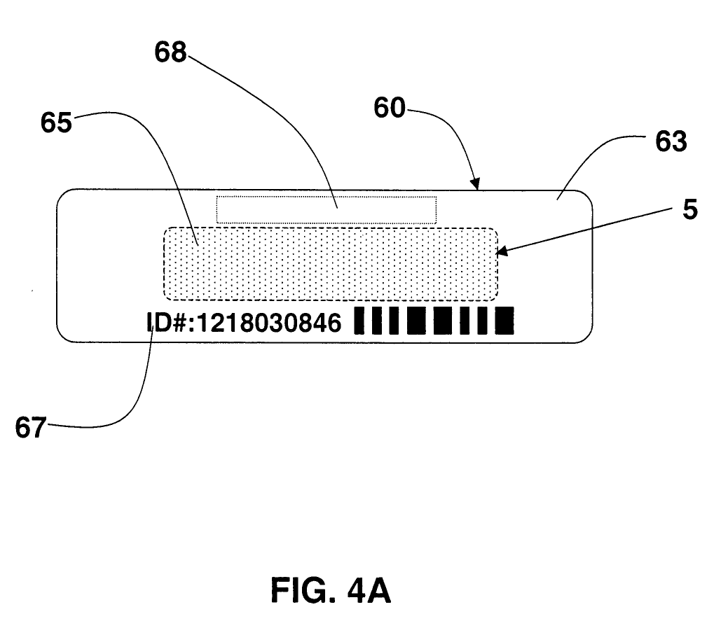

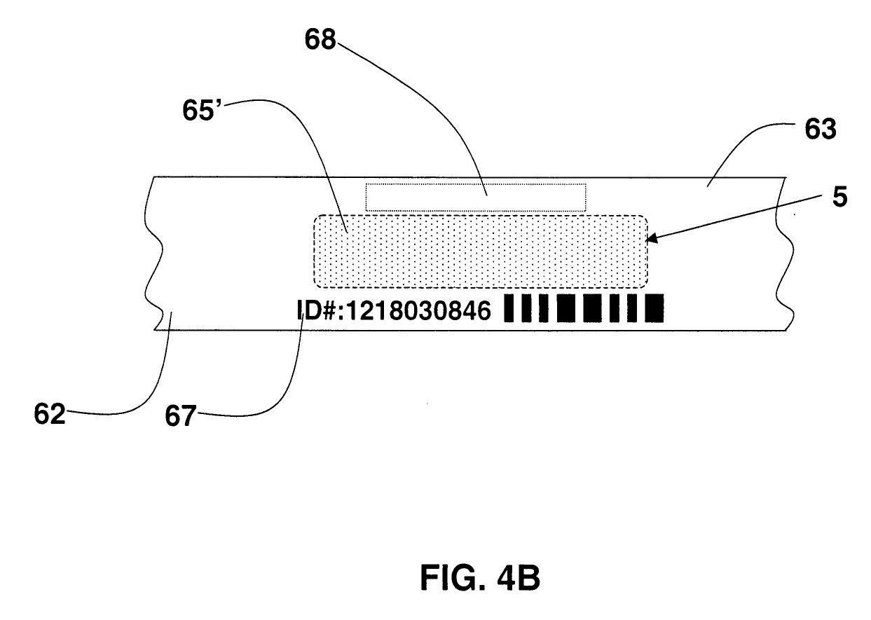

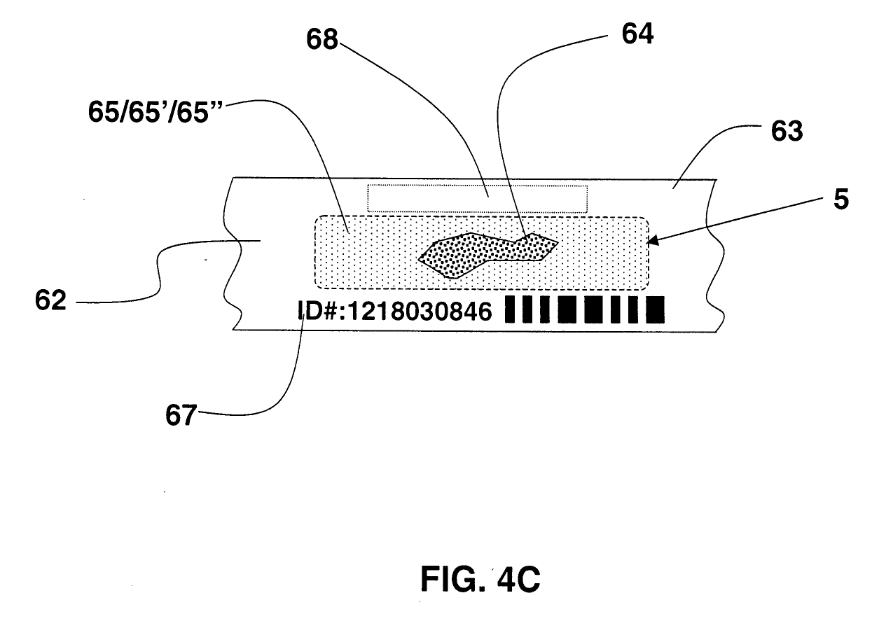

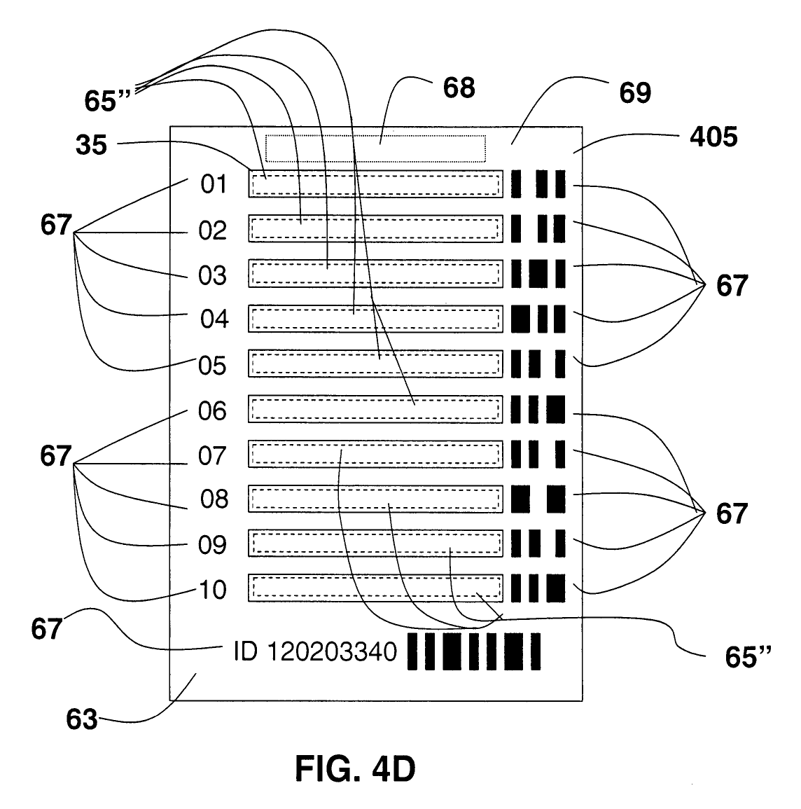

The sensor comprises a support, a sampling layer, and a signal amplification layer comprising silver halide. Referring to FIG. 1, there is illustrated a cross-sectional view of a multilayer sensor 5, which in the embodiment illustrated, comprises a support layer 10 with a signal amplification layer 15 comprising silver halide coated on the top surface 18 of the support layer 10 and a sampling layer 20 coated on the top surface 22 of the signal amplification layer 15. The sampling layer and the signal amplification layer may be the same layer, and this invention is intended to include such an embodiment. In such an embodiment the silver halide grains and the reactive material of the sampling layer may be blended homogeneously or may be regionalized. Generally the sensor is in the form of a test strip 60 having a sampling area 65 shown in FIG. 4A, however, in another embodiment the sensor may be a sampling area 65′ located on a web 62 as shown in FIG. 4B or sampling area 65″ may be located on a test strip array 69 as shown in FIG. 4D.

The sampling layer is able to react with a target species (pathogens, etc.) to form or release a signal compound, which can effect a reaction with the silver halide to form a latent image. Examples of pathogens include viruses, bacteria, etc. The sampling layer contains an interactive material, which reacts with the target species to form, or release a signal compound as described below. The target species may cause the sampling layer to release the signal compound or the signal compound may be formed through a reaction between the target species and a component of the sampling layer, either through a single reactive step or through a chemical cascade. The signal compound may effect the reaction with silver halide either by itself diffusing to the silver halide layer or through a chemical cascade through intervening layers. The signal compound can effect a direct reaction with the silver halide to form a latent image, or it can react with a secondary compound contained in the silver halide layer, which can then react with the silver halide to form a latent image. The interaction of the pathogen, signal compounds and silver halide are described in commonly-assigned copending U.S. Patent Application Publication No. 2005/0123440 (Switalski et al.) and U.S. Patent Application Publication No. 2005/0123439 (Patton et al.), which are incorporated herein by reference.

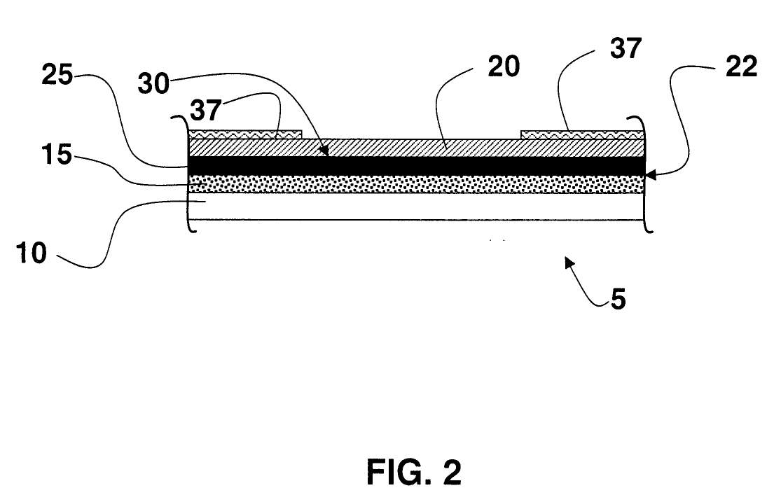

In one embodiment the multilayer sensor 5 further comprises a light-blocking layer 25 which blocks electromagnetic radiation, which is capable of exposing the silver halide. One embodiment made in accordance with the present invention is shown in FIG. 2, wherein the additional light-blocking layer 25 is coated on the top surface 22 of the signal amplification layer 15. If such a layer is not present the multilayer sensor 5 may have to be protected from light or other exposing radiation by some other means, such as being stored and utilized in some type of light-blocking container. The electromagnetic radiation which must be blocked will be dependent on the type of silver halide utilized and the method of sensitization utilized; for example, it may block all visible light, or only a portion of the visible spectrum. It may also only be necessary that ultraviolet light be blocked. The purpose of the light-blocking layer 25 is to prevent accidental and unintended exposure of the silver halide. In FIG. 2 the sampling layer 20 is coated on the top surface 30 of the light-blocking layer 25. An additional anti-microbial layer 37 maybe placed on over the sampling layer 20 allowing portions of the sampling layer 20 to be exposed.

In the embodiment, shown in FIG. 2, the light-blocking layer is positioned between the sampling layer and the silver halide layer. In this embodiment, wherein the light-blocking layer is between the sampling layer and the silver halide layer, the signal compound is capable of effecting a reaction with the silver halide by reacting with the light-blocking layer to effect a reaction with silver halide to form a latent image. The signal compound may react with a component in the light-blocking layer either through a single reactive step or through a chemical cascade. In another embodiment the sampling layer 20 is located between the light-blocking layer 25 and the silver halide layer. In yet another embodiment the sampling layer also blocks electromagnetic radiation, which is capable of exposing the silver halide, i.e., the sampling layer and the light-blocking layer are the same layer.

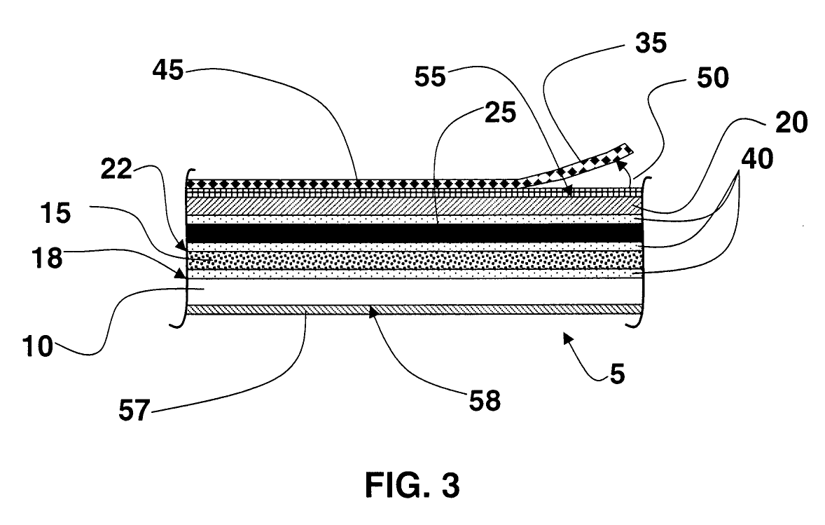

The support to be utilized is preferably opaque. In some instances, however, the support may be transparent in which case an additional blocking layer 57 shown in FIG. 3 may be coated on the bottom surface 58 of the support layer 10. The support layer may comprise any of the materials known in the art.

The sensor can contain additional layers, such as filter layers, interlayers, overcoat layers, subbing layers, and the like. The filter layer could be coated above the sampling layer to prevent interference materials from reaching the sampling layer or above the amplification layer to prevent interference materials from reaching the amplification layer, i.e., allowing only the signal compound to reach the amplification layer. Now referring to FIG. 3, there illustrates a cross section of yet another embodiment the multilayer sensor 5 of FIG. 1 made in accordance with the present invention. In the embodiment illustrated in FIG. 3 the multilayer sensor 5 comprises a removable protective layer 35 over the sampling layer 20. In some instances an additional release layer 45 may be required between the removable protective layer 35 and the sampling layer 20. Depending upon the material chosen for the support layer 10, an additional layer called a subbing layer 40 may be coated on the top surface 18 of the support layer 10. The subbing layer 40 is used to insure proper adhesion of the signal amplification layer 15 to the support layer 10. Likewise the subbing layer 40 maybe coated on the top surface 22 of the signal amplification layer 15. The subbing layer 40 is used to insure proper adhesion of a light-blocking layer 25 to the signal amplification layer 15. As previously discussed depending on what material is used for the support layer 10, the amplification layer 15 and the light-blocking layer 25, the subbing layer 40 may or may not be required. The addition of a subbing layer may or may not be required between any the adjacent layers of the multilayer sensor 5. Preparing a support surface (hydrophobic) such as polyvinyl alcohol to accept a solvent cast polymer such as cellulose triacetate would require chemical and/or an interlayer coating (subbing layer) to improve adhesion. An example of this could be found in photographic patent literature where gelatin based hydrophilic photographic materials are commonly attached to hydrophobic supports such as polyethylene terephthalate.

In the embodiment illustrated in FIG. 3, an optional peelable protective layer 35 and the release layer 45 may be combined and provided over the sampling layer 20 for protecting the sampling layer 20 until the multilayer sensor 5 is to be used for testing. The combination protective layer and release layer is peeled off the sampling layer 20 as indicated by arrow 50 exposing the top surface 55 of the sampling layer 20.

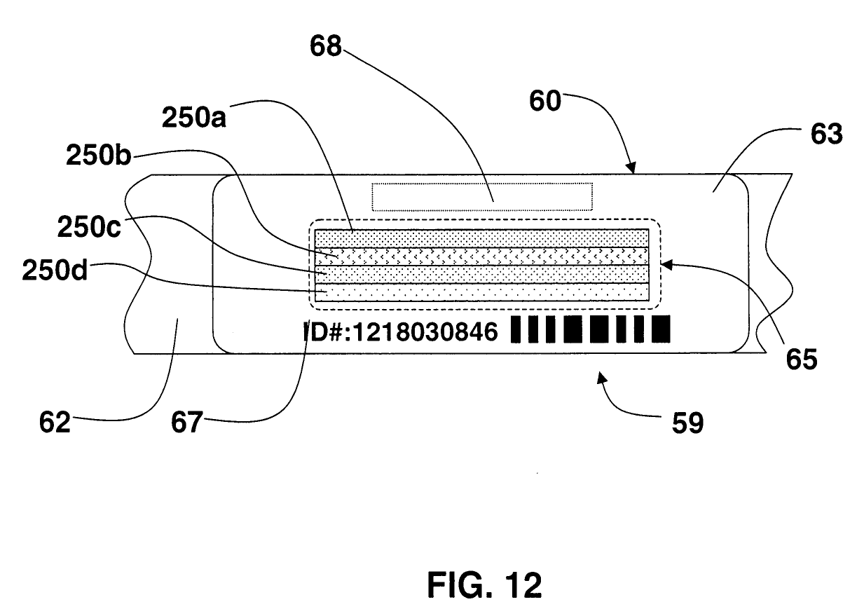

In one embodiment the sensor can detect more than one type of disease. In one suitable embodiment the sampling layer would be striped as shown in FIG. 12 with each stripe being sensitive to a different target species. As can be appreciated, a variety of different elements, depending on the analysis of choice, can be prepared in accordance with the present invention. Sensors can be configured in a variety of forms, including elongated tapes of any desired width, sheets or smaller chips. As noted above, test strips are particularly contemplated.

In the case of the mouth or throat sensor, for example, the mouth or throat is swabbed for suspected bacteria or virus. The swab is applied to the sensor. At very low concentrations, the sampling layer would release chemistry (e.g., LIFCS) that would diffuse to the silver halide layer, causing a latent image. This latent image is amplified when the sensor is either developed by a triggerable chemistry, or with heat. The silver halide may form a black and white image or the development of the silver may result in chemistry, which develops uncolored compounds (known as couplers) to colored dyes. The colors are observed and recorded. They can be “stopped” or “fixed” at any point, can be scanned for density to obtain a quantitative number, and can be stored or catalogued for later use (confirmation, verification, audit, etc.). Black and white processing methods are well known in the art.

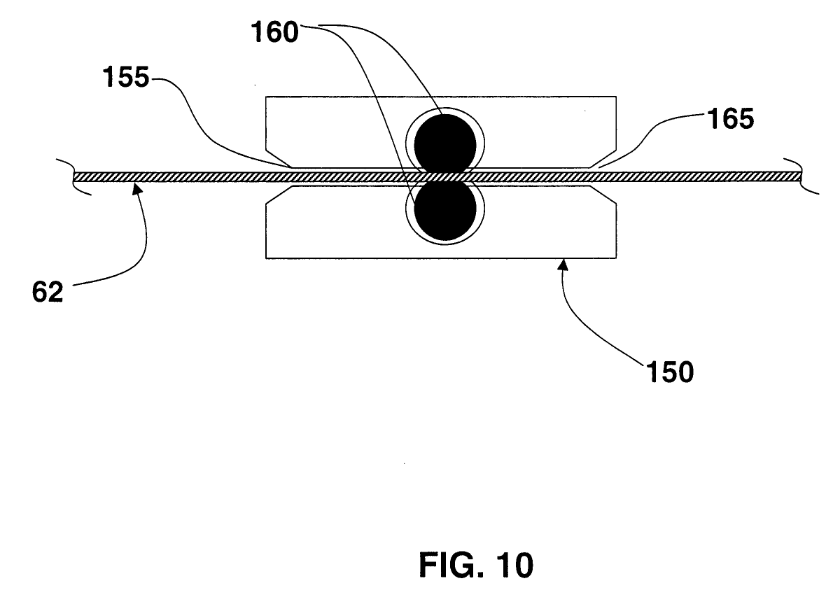

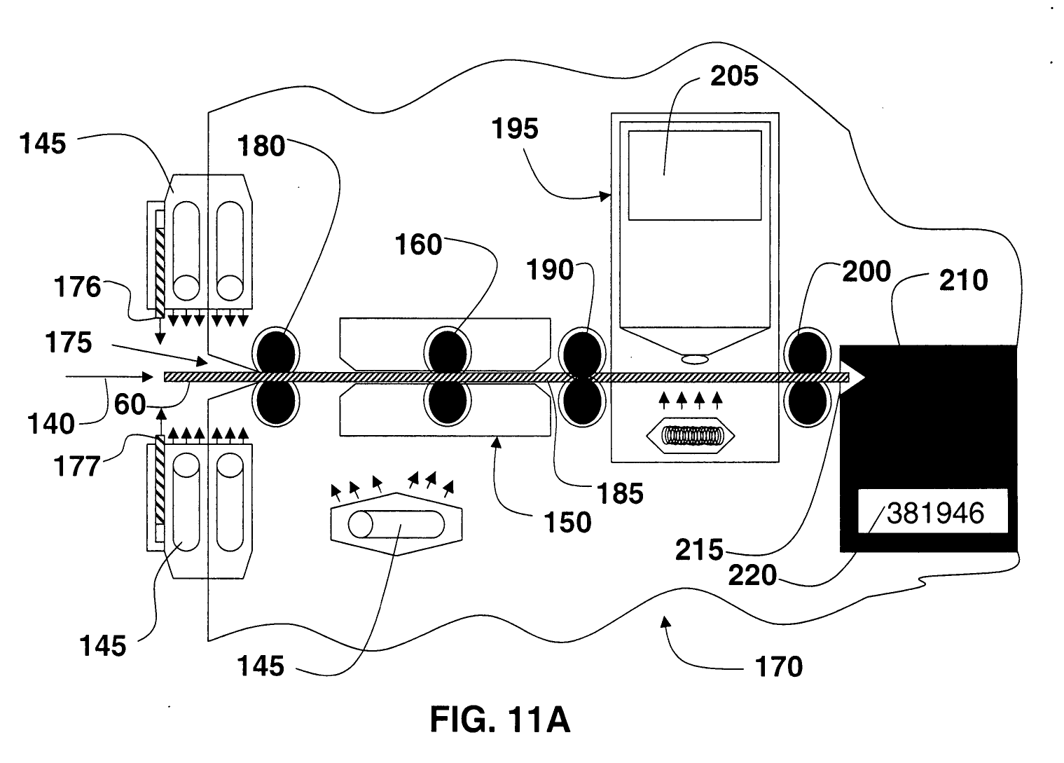

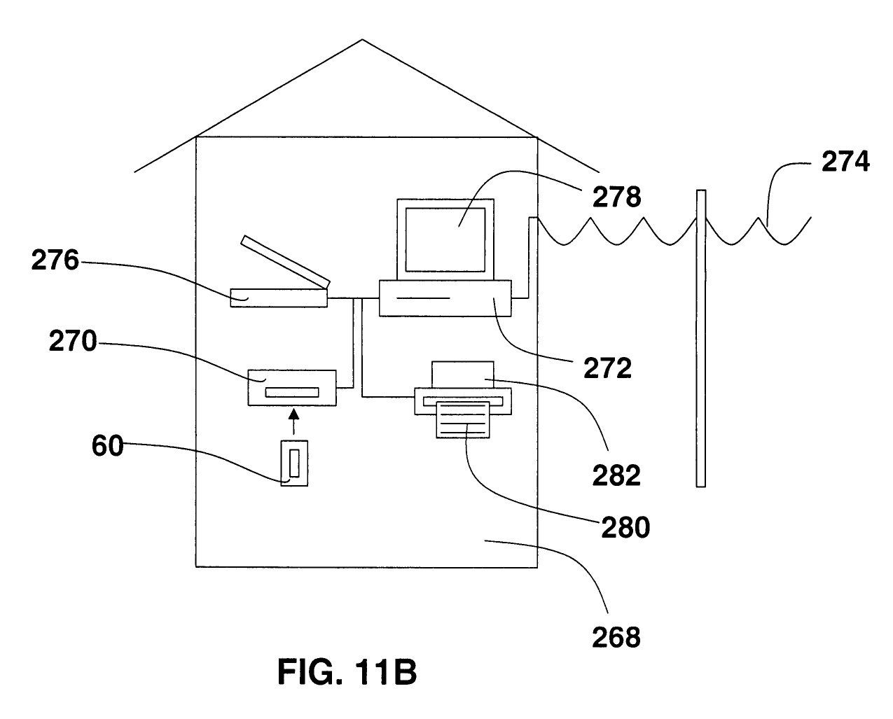

The preferred method of development involves the use of heat with a thermally sensitive silver emulsion similar to a photothermographic material. Heat processing devices are described later in FIGS. 10 and 11. If the sensor is to be heat developed the silver halide amplification layer will generally comprise silver halide that upon LIFCS exposure provides a latent image in exposed grains that are capable of acting as a catalyst for the subsequent formation of a silver image in a development step, (b) a non-LIFCS sensitive source of reducible silver ions, (c) a reducing composition (usually including a developer) for the reducible silver ions, and (d) a hydrophilic or hydrophobic binder. The latent image is then developed by application of thermal energy.

The thermally developed materials of the invention can also contain other additives such as shelf-life stabilizers, antifoggants, contrast enhancing agents, development accelerators, acutance dyes, post-processing stabilizers or stabilizer precursors, thermal solvents (also known as melt formers), humectants, and other image-modifying agents as would be readily apparent to one skilled in the art.

Thermal development conditions will vary, depending on the construction used but will typically involve heating the LIFCS exposed material at a suitably elevated temperature. Thus, the latent image can be developed by heating the exposed material at a moderately elevated temperature of, for example, from about 50° C. to about 250° C. (preferably from about 80° C. to about 200° C. and more preferably from about 100° C. to about 200° C.) for a sufficient period of time, generally from about 1 to about 120 seconds. Heating can be accomplished using any suitable heating means such as a resistive heater, hot plate, a steam iron, a hot roller, mechanical finger or a heating bath. A preferred heat development procedure includes heating at from about 110° C. to about 135° C. for from about 3 to about 25 seconds. One can also use a light source, such as a laser beam, that is absorbed by any portion of the layered structure, but preferably the layer containing the latent image to develop, and preferably a wavelength that can be matched to absorb best in this layer without unwanted development, such as a near-infrared or infrared wavelength supplied by a near-infrared or infrared laser diode.

After the sensor has been processed using any of the methods described above or using a conventional photographic processor, the sensor may be electronically scanned. The scan may then be digitized and analyzed using a computer (not shown) and the results of the computer analysis outputted via a printer or displayed electronically. The results of several individual sensors may be compared.

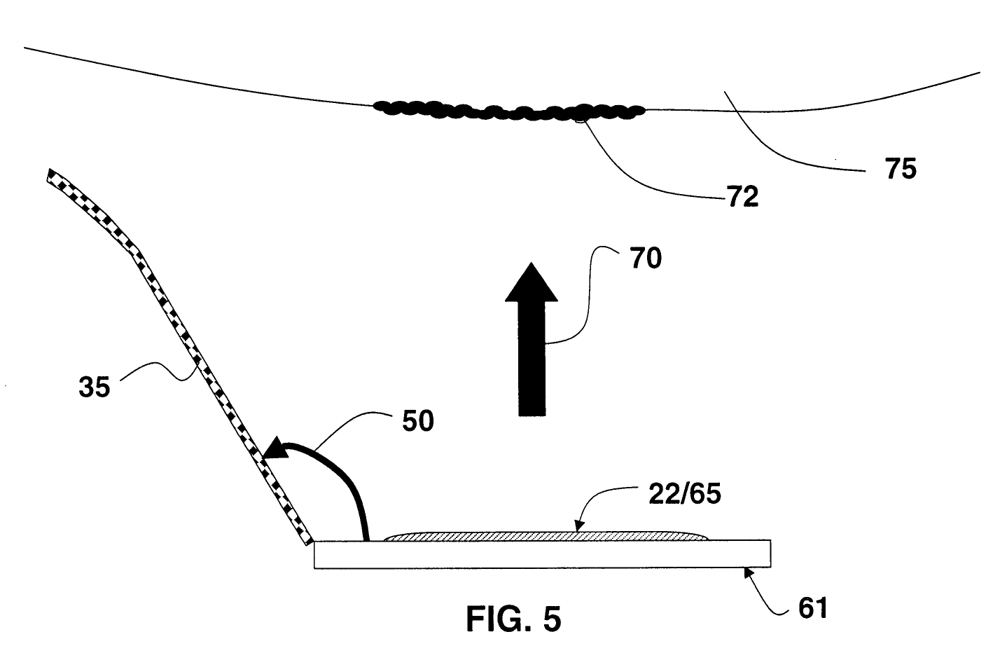

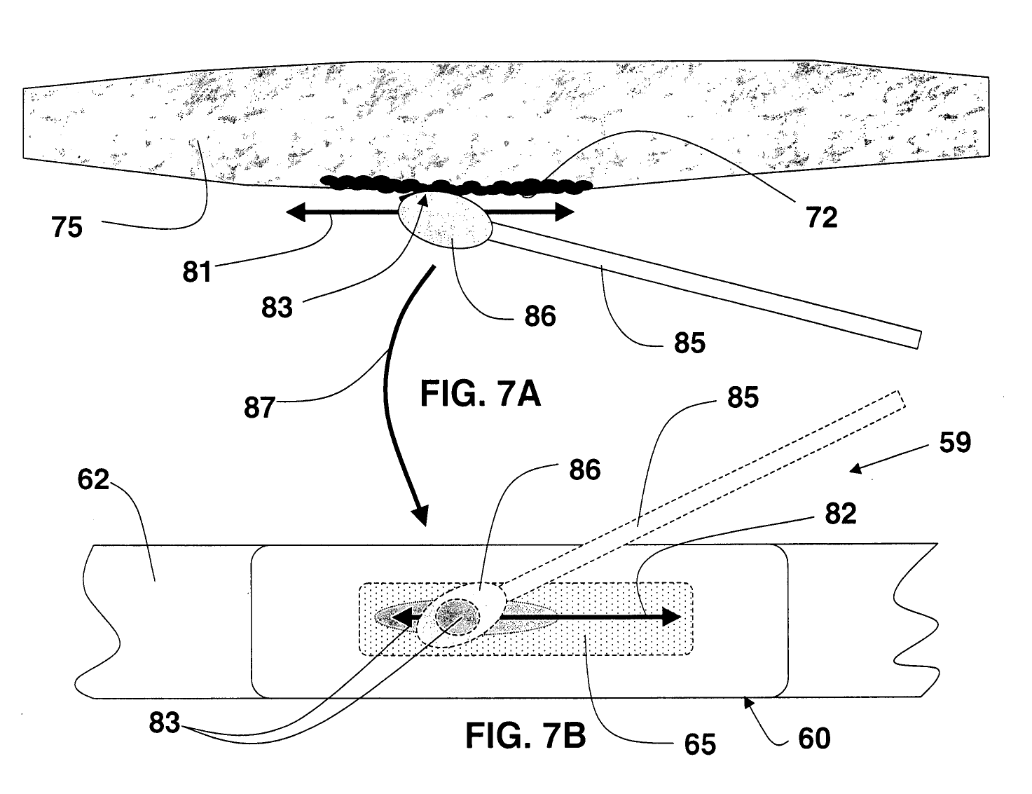

As noted above it is contemplated that the multilayer sensor 5 may be part of a test kit 59. In the embodiment shown in FIG. 4A, the multilayer sensor 5 is in the form of a test strip 60. The test kit 59 including the test strip 60 may also provide for several ways to obtain a sample such a sampling patch 61 shown in FIGS. 4A and 5 or a swab 85 shown in FIGS. 7A and 7B. Referring now to FIGS. 4A and 4B, the test strip 60 or web 62 comprises a support layer 10 containing a non-sampling area 63, and a sampling area 65 or 65′, which is comprised of the multilayer sensor 5 illustrated in FIGS. 1, 2, and 3. The non-sampling area 63 is used, for example, to provide ease of handling, space for printed data 67, both eye readable and machine readable, such as a unique identification number, and a writeable area 68. The non-sampling area 63 may surround the sampling area or the sampling area may be in a discrete portion of the test strip such as along the edge portion so the non-sampling area may be used to hold the strip. The non-sampling area 63 may contain an AgX anti-microbial material, compound or composition such as described in commonly-assigned U.S. Pat. No. 6,689,335 (Bringley et al.); U.S. Patent Application Publication Nos. 2005/0129742 and 2005/0129766 (both to Bringley et al.), hereby included by reference. The anti-microbial material will kill and prevent future growth of any pathogens that accidentally land on the non-sampling area 63. The optionally removable protective layer 35 covers at least the sampling area 65 and protects the sampling area 65 (shown as a hidden view) until the test strip 60 is to be used. The protective layer may be light-blocking as described above for the light-blocking layer. The test strip may also comprise a release layer 45. FIG. 4C illustrates the sampling area 65 or 65′ of the multilayer sensors of FIGS. 4A and 4B with a sample 64 present on the sampling area 65 or 65′.

Now referring to FIG. 4D, there is illustrated yet another embodiment in the form of a test strip array 69, comprising a non-sampling area 63 and multiple sampling areas 65″ shown. Each sampling area may be uniquely identified with prerecorded data. The test strip array may allow for multiple testing of different test areas for the same target species (i.e. the sampling areas to detect the same target species) or multiple testing of one test area or more for a variety of different target species (i.e. the sampling areas can detect different target species). The non-sampling area is used again, for example, to provide ease of handling, space for printed data 67, (prerecorded data) both eye and machine readable, such as an identification number, and a writeable area 68. The test strip array may comprise one or more than one removable protective layers 35, each protecting a sampling area 65″ (shown as a hidden view) or a portion of the sampling areas until the individual or group of sampling areas is to be used. It may also comprise one protective layer covering the entire test strip array. In one embodiment, the protective layer may be removed and replaced at various times after exposure. It may be replaced after the sample is applied to the test strip array to protect the sampling area during transport, handling or while other samples are being applied. It may also be replaced prior to heat processing allowing the results of the test to be viewed while keeping the sampling area 65″ from being contaminated. It also prevents the user 305 from touching the sampling area 65″ and being exposed to the pathogen. As previously described for the test strip, the test strip array may be viewed through the base by removing protective layer 35, not shown. Blocking layer 57 is removable. The blocking layer 57 is peeled away exposing the bottom surface of the support layer 10 through which the signal amplification layer 15 of the multilayer sensor 5 is visible. In one embodiment the blocking layer is light-blocking as described above; however, it need not be diffusible.

The method of using the test strip array is generally the same as described for the test strip. Because of the multiple test areas it is generally used with a transfer device such as the swab. A test area is contacted with a transfer device and the transfer device is placed in contact with one or more sampling areas of the test strip array. In one embodiment multiple test areas are contacted with separate transfer devices and each transfer device is placed in contact with one or more sampling areas of the test strip array. In another embodiment the same test area is contacted with separate transfer devices and each transfer device is placed in contact with one or more sampling areas of the test strip array.

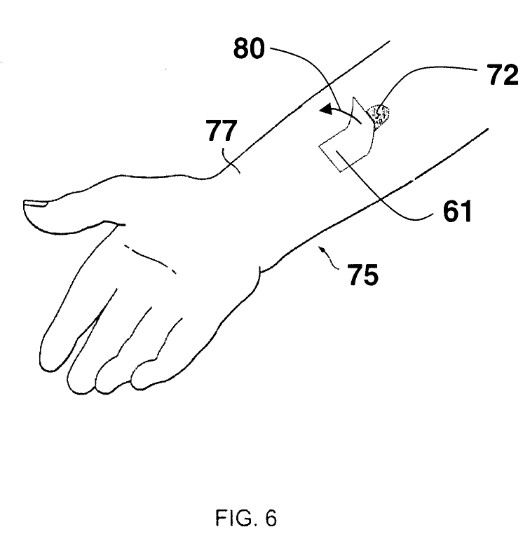

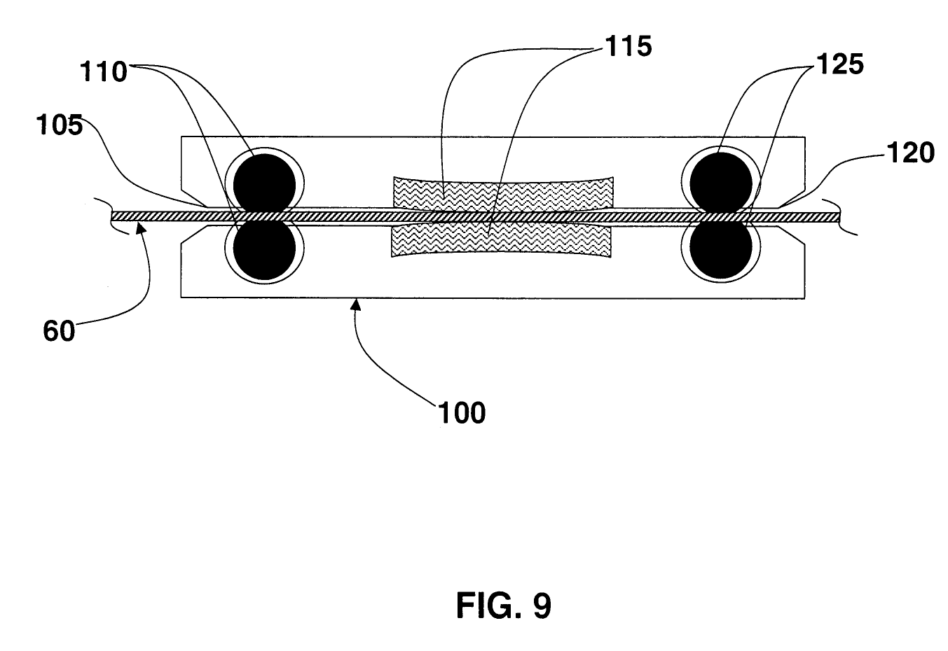

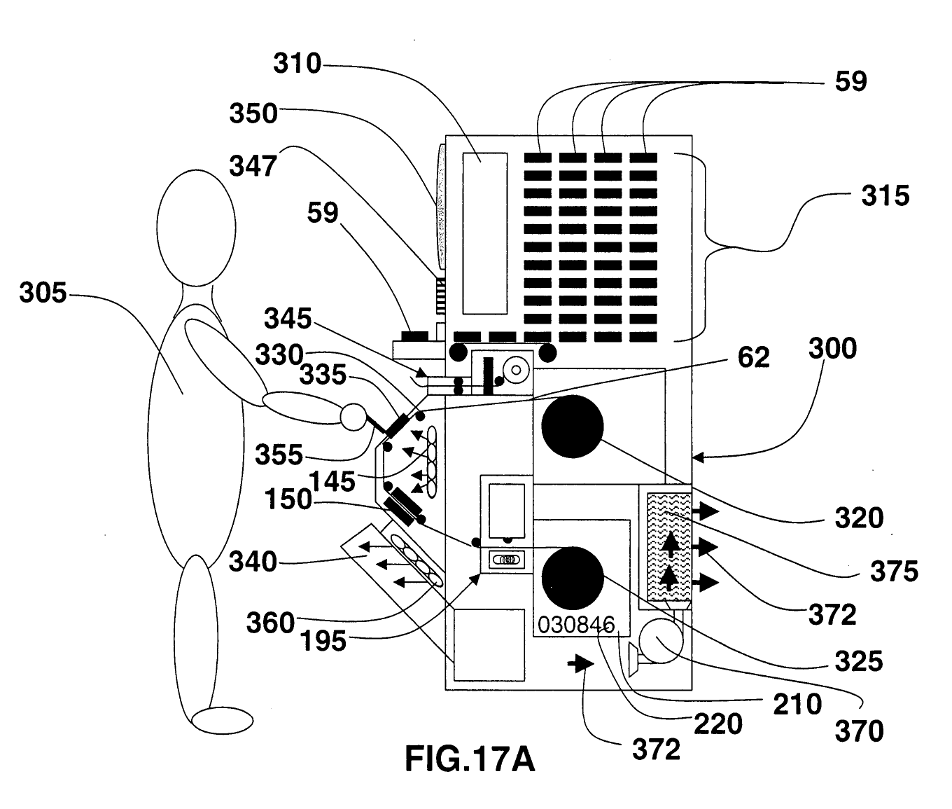

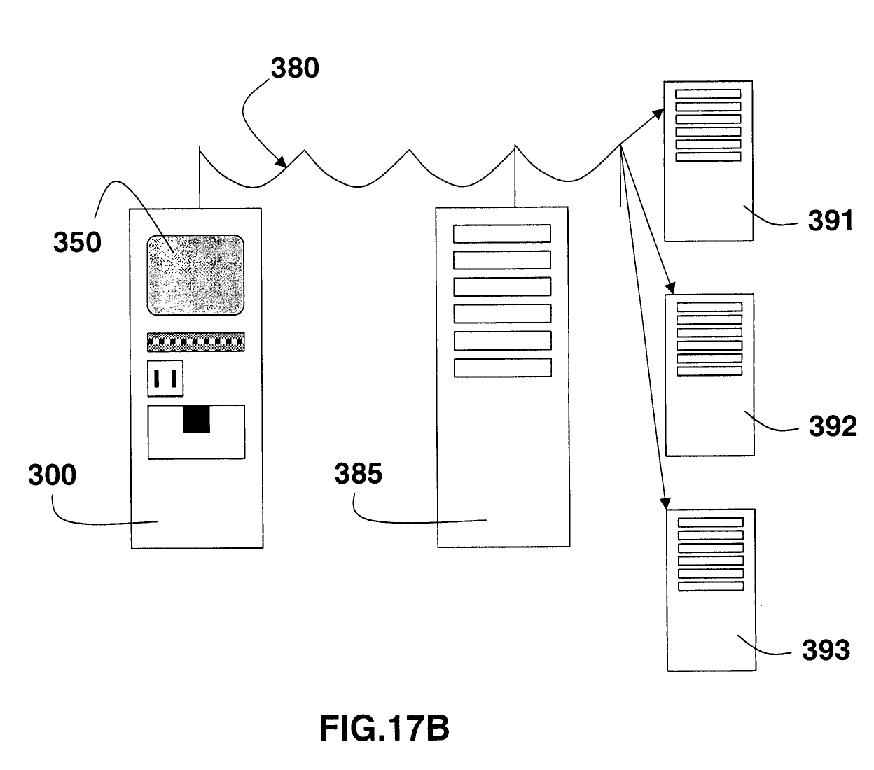

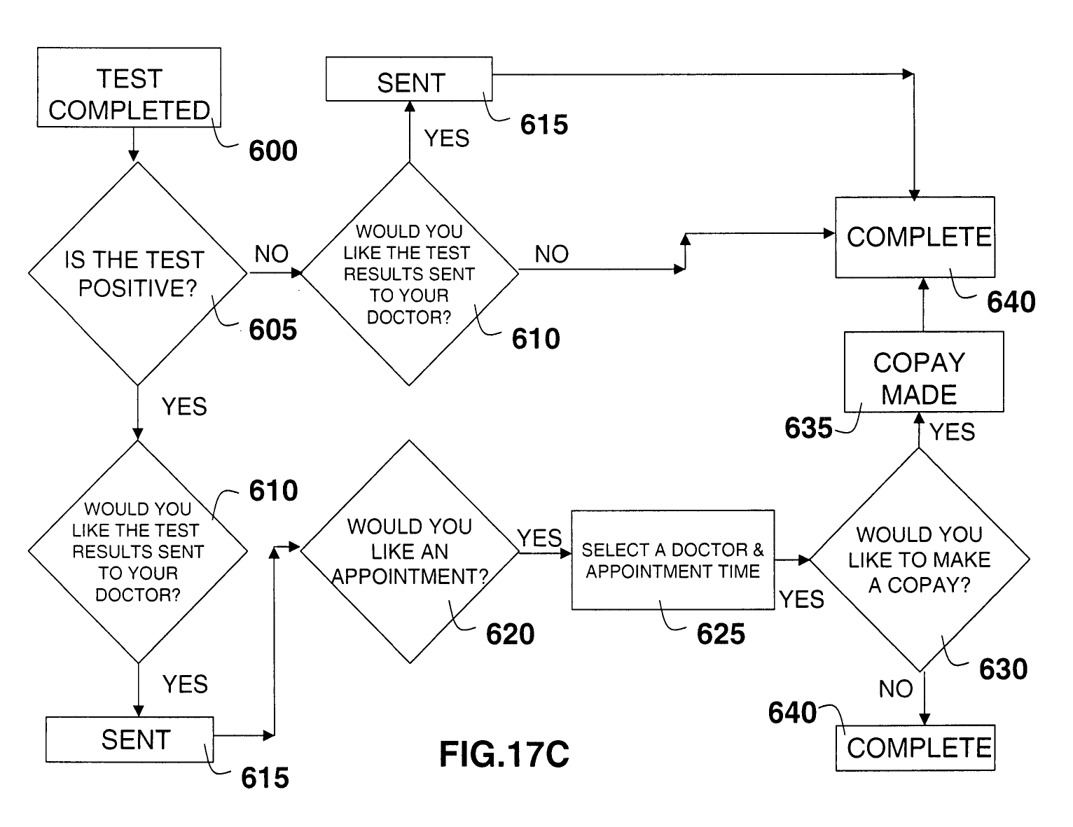

In the embodiment shown in FIGS. 5 and 6, the test strip 60 is in the form of a sampling patch 61. In this embodiment the sample 64 (shown in FIG. 4C) is obtained by peeling the optionally removable protective layer 35 off the sampling patch 61 as indicated by arrow 50 and exposing the top surface of the sampling area 65 (i.e. the sampling layer 20 of the multilayer sensor 5). The sampling patch 61 is then firmly placed as indicated by arrow 70 against the test area 75, for example a suspect area 72 on a person's skin 77 such as on an arm, bringing into direct contact the sampling area 65 with suspect area 72. The sampling patch 61 is then peeled from the test area 75 as indicated by arrow 80 in FIG. 6 with the sample 64 on the sampling area 65. The sampling patch 61 is then developed as described below. In this embodiment the removable protective layer 35 may be replaced prior to processing, allowing the results of the test to be viewed while keeping the sampling area 65 from being contaminated. It also prevents the user 305 (shown in FIG. 17A) from touching the sampling area 65 and being exposed to the sample area. The removable protective layer 35 may be transparent allowing scanning after processing as described later in FIG. 9.

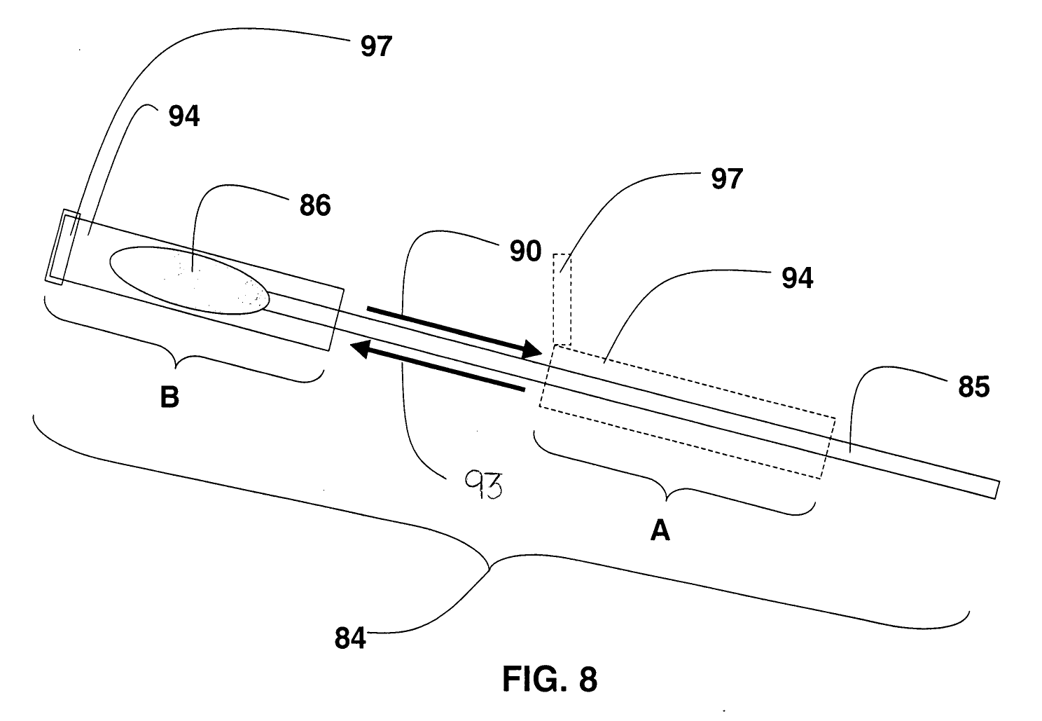

In another embodiment as shown in FIGS. 7A and 7B, a kit comprising a transfer device such as a swab maybe used to collect a sample from the inside of the mouth, throat, eye, etc. The suspect area 72 of the test area 75, for example the throat or mouth, is swabbed as indicated by arrow 81. The sample 83 present on the swab sampling tip 86 is applied to the sampling area 65 or 65′ of either the test strip 60 or the web 62 respectively as indicated by arrow 87 in the direction indicated by arrow 82 respectively. Now referring to FIG. 8, there is illustrated a swab assembly 84 comprising the swab 85 and a protective enclosure 94 with an enclosure cap 97 covering the swab sampling tip 86. When the swab 85 is used the enclosure cap 97 is opened and the protective enclosure 94 is slid down the swab 85 to position “A” as indicated by arrow 90. After the swab 85 has been used to take a sample, the protective enclosure 94 is slid up the swab 85 to position “B” as indicated by arrow 93 and the enclosure cap 97 is closed covering the swab sampling tip 86. The protective enclosure 94 is used to prevent the swab 85 from being contaminated while preventing the user 305 (shown in FIG. 17A) from touching the swab 85 and being exposed to the swab sampling tip 86. At very low concentrations, the sampling layer would release chemistry (e.g., LIFCS) that would diffuse to the silver halide layer, causing a latent image. This latent image is amplified when the sensor is either developed by a triggerable chemistry, or with heat. The development of the silver may result in a black and white image or in chemistry, which develops uncolored compounds (known as couplers) to colored dyes. The black and white image or colors are scanned, and recorded. They can be “stopped” or “fixed” at any point, can be scanned for density to obtain a quantitative number, and can be stored or catalogued for later use (confirmation, verification, audit, etc).

In general, the sampling area 65 of the test strip 60 is contacted with the material from the suspect area 72 to be tested and the silver halide image is allowed to form a latent image. The latent image is then developed to form a detectable signal. The signal may be an on/off signal or it may be measurable to indicate the amount of the target species present. The latent image may be developed by heat or by chemical processing. The signal is then read visually or by a densitometer, gas chromatograph mass spectrometer, or a scanning device such as KODAK PROFESSIONAL HR Universal Film Scanner as described later. The test strip may be placed in contact with the suspect area of the test area or the suspect area may be contacted with a transfer device and the transfer device containing the test material placed in contact with the test strip or web.

Black and white processing methods are well known in the art. One method of development involves the use of heat (shown in FIGS. 9 and 10) with a thermally sensitive silver emulsion. The source of heat can be a resistive heater; a heated platen, roller, or mechanical finger; or a light source, such as a laser beam. Now referring to FIG. 9, there is described a method for processing the test strip 60 using a heat processor 100. The heat development is the same as is used in dry film development systems, such as the Kodak DryView X-ray film system. The test strip 60 is fed through the heat processor 100 via an entry port 105 and driven by a pair of entry drive rollers 110 through two heating platens 115 and out through and exit port 120 by a pair of exit drive rollers 125.

A second embodiment of a heat processor 150 is illustrated in FIG. 10. In this embodiment the web 62 with the test sample 64 on the sampling area 65′ (shown in FIG. 4D) is fed into the heat processor 150 via an entry port 155 and is conveyed via a pair of heated rollers 160 and exits through an exit port 165. Again the heat development is the same as is used in dry film development systems, such as the Kodak DryView X-ray film system.

After the user 305 has sampled the suspect area 72, the user 305 recovers the sampling area 65 with the removable protective layer 35 and places the test strip 60 into the apparatus 170 shown in FIG. 11A via an entry slot 175. After the test strip 60 entered the apparatus 170, the entry slot doors 176 and 177 close and the test strip 60 is processed, the entry slot 175 and interior are sterilized via a UV light source 145. The apparatus 170 may be incorporated into a kiosk 300 described later in FIG. 17A. Still referring to FIG. 11A, the test strip 60 is fed into the heat processor 150 as indicated by arrow 140 by a pair of entry drive rollers 180 and driven through the heat processor 150 by the pair of heated rollers 160 and out through and exit port 185 by a pair of exit drive rollers 190. After being processed, the test strip 60 is transported through the scanner 195 via the exit drive rollers 190 and scanner drive rollers 200 to allow the resulting processed image (not shown) to be scanned via the scanner 195 and stored in the microprocessor 205. The processed and scanned test strip 60 is then fed into the storage and/or disposal box 210 via entry port 215. The disposal box 210 may be used to safely store the processed and scanned test strips 60 for later collections. Because each strip is uniquely identified by the unique identification number 67, the strips may be later retrieved for reexamination and/or rescanning. The disposal box 210 may also be used as a container for safe disposal of the test strips as medical waste. Each disposal box may have a unique identification number 220 linking it to a specific kiosk 300 or site.

Referring to FIG. 11B, the apparatus 170 described in FIG. 11A may be used by the user in the home 268 by incorporating apparatus 170 into a home diagnostic unit 270 that attaches directly to the user's home computer 272 and may communicate with a trained technician, doctor's assistant, nurse practitioner, pharmacist, doctor or specialist via the user's Internet connection 274. The user purchases the test kit 59 and carries out the desired test as described above. If the home diagnostic unit 270 does not have the scanner 195, the user may scan the processed test strip 60 via the user's personal scanner 276 attached to the user's computer 272. The results are then transmitted to the doctor via the Internet. The results are then transmitted back to the user along with the doctor's instructions and displayed on the computer's monitor 278. The user may print out the results and instructions 280 on the user's home printer 282.

In yet another embodiment, as illustrated in FIG. 12, the multilayer sensor 5 can detect more than one type of disease. The sampling layer 20 of the sampling area 65 of either the test strip 60 or the sampling area 65′ of the test web 62 may be designed with multiple coatings, so that test areas 250a, 250b, 250c, and 250d of the strip are selective to different suspect pathogens. In the embodiment shown the test areas 250a, 250b, 250c, and 250d are shown running parallel to printed data 67. The test areas however may also run in a direction perpendicular to printed data 67.

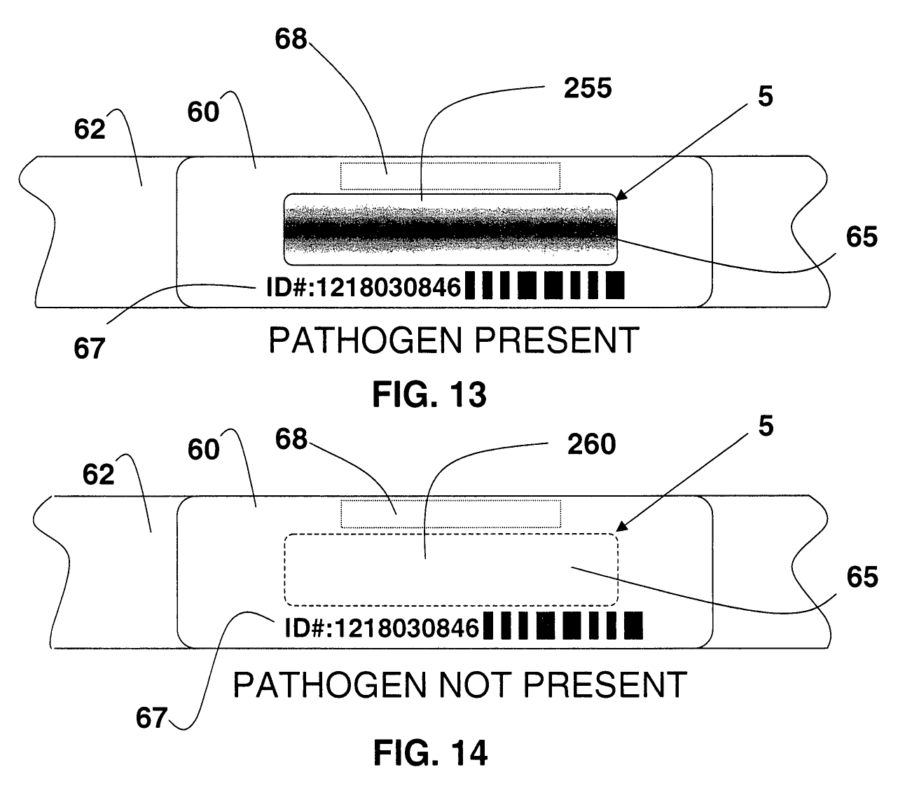

Now referring to FIGS. 13 and 14, the results of processing the test strip 60, the test web 62 (multilayer sensor 5 in the form of a web), or the test array 69 using the methods described above in FIG. 9 or 10 are shown. If the test for the pathogen is positive, i.e. the pathogen was present, the sampling area 65, 65′, or 65″ would darken with an increase in density similar to the density of an image on a black and white film negative or change color as shown in FIG. 13. In the example shown the test area has density as indicated by 255. If the test for the pathogen is negative, i.e. the pathogen was not present, the sampling area 65, 65′, or 65″ would not darken or change color as shown in FIG. 14. In the example shown the test area remains unchanged as indicated by 260.

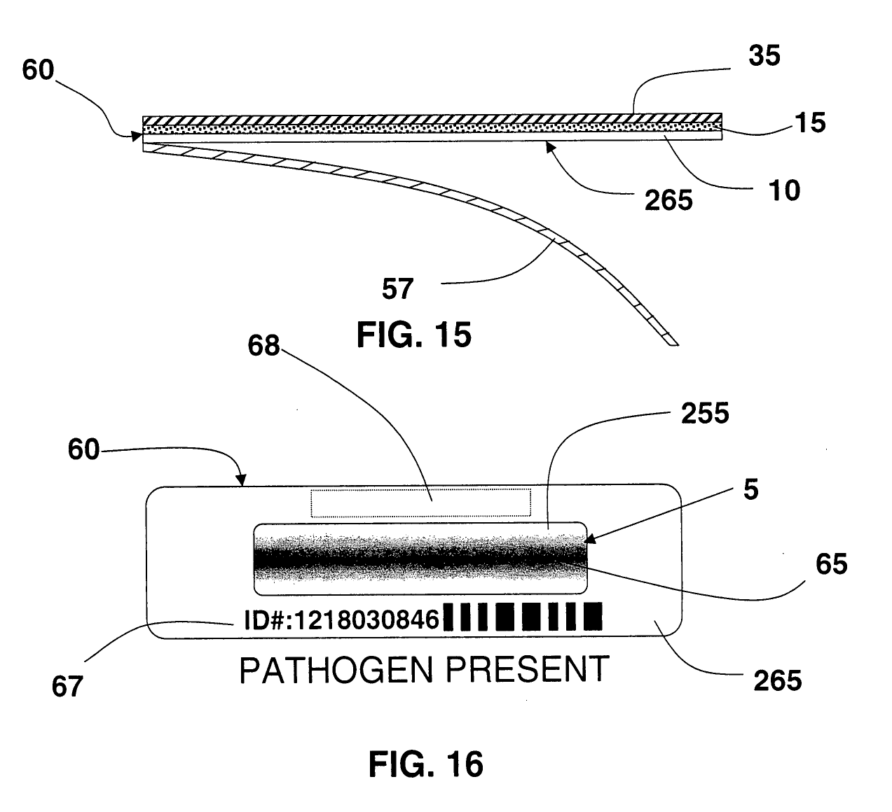

Now referring to FIG. 15, there is illustrated another embodiment of the test strip 60 (multilayer sensor 5) processed using the methods described above in FIG. 9 or 10. In this embodiment the blocking layer 57 is located on the opposite side of the support layer 10 of the test strip 60 from the signal amplification layer 15. Blocking layer 57 is removable. The blocking layer 57 is peeled away exposing the bottom surface 265 of the support layer 10 through which the amplification layer 15 of the multilayer sensor S is visible as shown in FIG. 15. In one embodiment the blocking layer is light-blocking as described above, however, it need not be diffusible. In the example shown the test area is darkened as indicated by 255 as illustrated in FIG. 16.

Now referring to FIG. 17A, there is illustrated a schematic of the kiosk 300 made in accordance with the present invention. The kiosk 300 comprises a microprocessor 310, test kit dispensing assembly 315, a test web 62, supply roll 320, and take up roll 325. The take up roll 325 is located in a disposal box 210 (as previously described in FIG. 11), a web transport assembly 330, a sample placement area 335, the heat processor 150, the scanner 195, a medical waste disposal unit 340, a printer unit 345, and a monitor 350. The monitor 350 may be a touch screen monitor for used for input information and instruction to the microprocessor unit 310. The kiosk may also have a card port 347 for inserting and using a debit/credit card, money, a medical identification card, license, passport, etc.

In the following example the user 305 walks up to the kiosk 300 and inserts his or her credit card and medical identification card into the card port 347. A driver's license or national identity card may be used. Using the touch screen monitor 350 the user 305 chooses what type of test his or she would like to use. In this case the user 305 chooses the test kit 59 for a strep throat bacteria infection test. The microprocessor 310 dispenses the appropriate test kit 59. Following the instructions displayed on the monitor 350 and previously described in FIGS. 7A, 7B, and 8, the user 305 opens the sterile test kit, removes the swab 85, and swabs his or her throat. The user then opens the sampling area door 355 on the kiosk 300 and rubs the swab 85 on the sample area 65′ of the web 62 as also previously describe in FIGS. 7A, 7B, and 8. The user 305 then closes the sample area door 355 causing the microprocessor 310 to transport the web 62 through the processor 150 and scanner 195. After the test web 62 or test strip 60 has been processed using any of the methods previously described, the test web 62 or test strip 60 may be electronically scanned. The scan may then be digitized and analyzed using the microprocessor 310 which may be a computer and the results of the analysis outputted via a printer 345 or displayed electronically via the monitor 350. The results may also be transmitted via a communications network to a central lab, doctor's office, etc. where the results may be further analyzed by a trained technician, doctor's assistant, nurse practitioner, pharmacist, doctor, or specialist. The results of the analysis may then be transmitted back to the kiosk 300 and displayed to or printed for the user 305 to read. Included in the results may be the script for a prescription, and/or instructions to go to a specific pharmacy where the notification has been received to fill the prescription. In the case where the remote diagnostic device is not connected to a communications network or connection to the communications network has been disrupted the script for a prescription may be printed out directly using the remote diagnostic device's or kiosk's printer 345 and dispensed to directly the user 305.

If the results indicate a positive result, the user may be offered the opportunity to make an appointment with the doctor or specialist. The kiosk 300 via a communications network 380 is connected to a server 385 as shown in FIG. 17B. The server 385 may contain and/or may be linked to one or more servers 391, 392 and 393, which contain information such as the user's health care provider, the user's personal doctor or doctor's, dockets who specialize in the diagnosis and treatment of the various aliments and diseases the kiosk is capable or diagnosing. The kiosk 300 may be directly linked via the communications network 380 to one or more servers 391, 392 and 393.

Referring now the FIG. 17C, when the test is complete step 600 and the user 305 receives a diagnosis step 605, the user 305 is asked whether or not the user would like the results sent to their doctor step 610. If yes, the results are sent step 615. If the diagnosis is positive, the user is asked if they would like to make an appointment with the doctor or specialist step 620. If the user 305 answers yes, the user then picks the doctor or specialist and the time and date for the appointment from a list of doctors and times step 625. The healthcare provider confirms the appointment and sends a referral to the doctor who has been chosen and a notification is sent to the user's personal doctor. The user is then asked if they would like to make the co-pay step 630. If the user answers yes the co-pay is made step 635, a receipt and appointment notification is printed for the user. If the user answers no, the transaction and test are completed step 640. If the diagnosis is negative the transaction and test are completed step 640.

The user 305 via the medical waste disposal unit 340 then disposes of the swab and the remains of the test kit 59. The kiosk may withhold displaying the results of the test on the monitor 350 or printing the results via the printing unit 345 until the user properly disposes of the waste in the medical waste disposal unit 340. It should be noted the kiosk may be equipped with privacy screens and the UV light source 360 to sterilize the surfaces, etc. To further insure cleanliness the kiosk 300 may be equipped with a fan 370 as shown in FIG. 17A, which draws air and indicated by the arrows 372 into the kiosk 300 and exhausts the air out of the kiosk 300 through a filter 375 containing the AgX anti-microbial material as previously described. The exhausted air as it passes through the filter 375 as indicated by arrow 372 is cleansed of pathogens by the AgX anti-microbial material. The filter 375 may be, for example, a Hepa^{R} Filter, which may be used to further filter out the pathogens from the exhausted air.

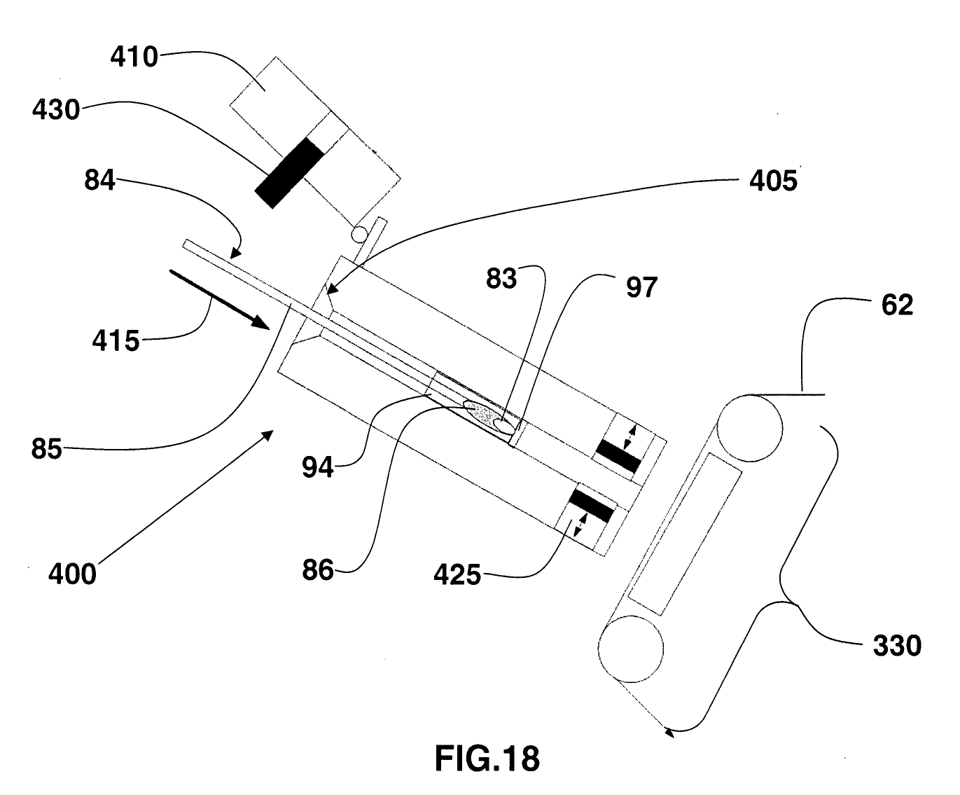

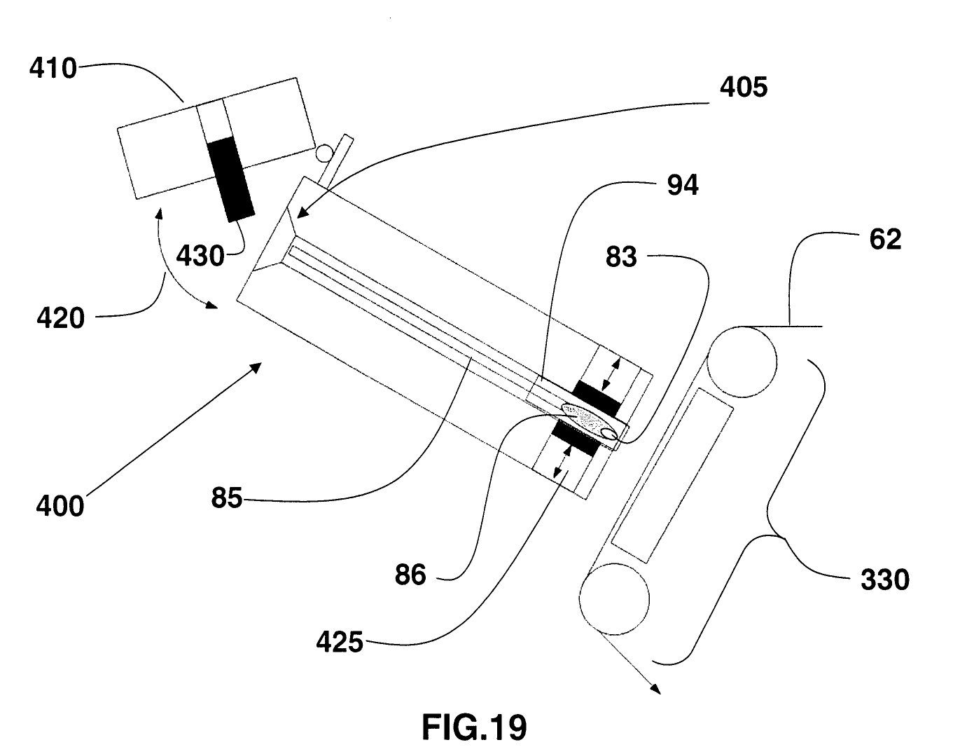

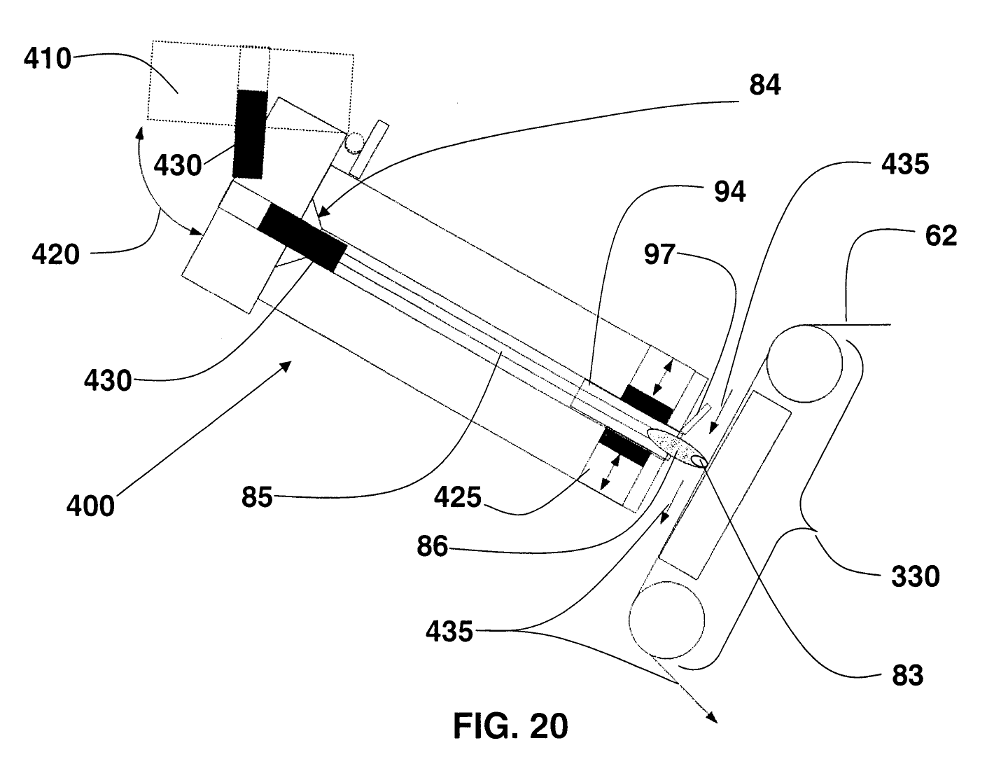

Now referring to FIGS. 18, 19, 20, and 21, there is illustrated a schematic of a transfer apparatus 400 used to apply the sample 83 from the swab sampling tip 86 to the sampling area 65′ (shown in FIG. 4B) on the web 62 in a kiosk 300 (shown in FIG. 17) without causing cross-contamination or contaminating the kiosk sample placement area 335. In the embodiment shown, the transfer apparatus 400 comprises a slot 405 for placing and holding the swab assembly 84.

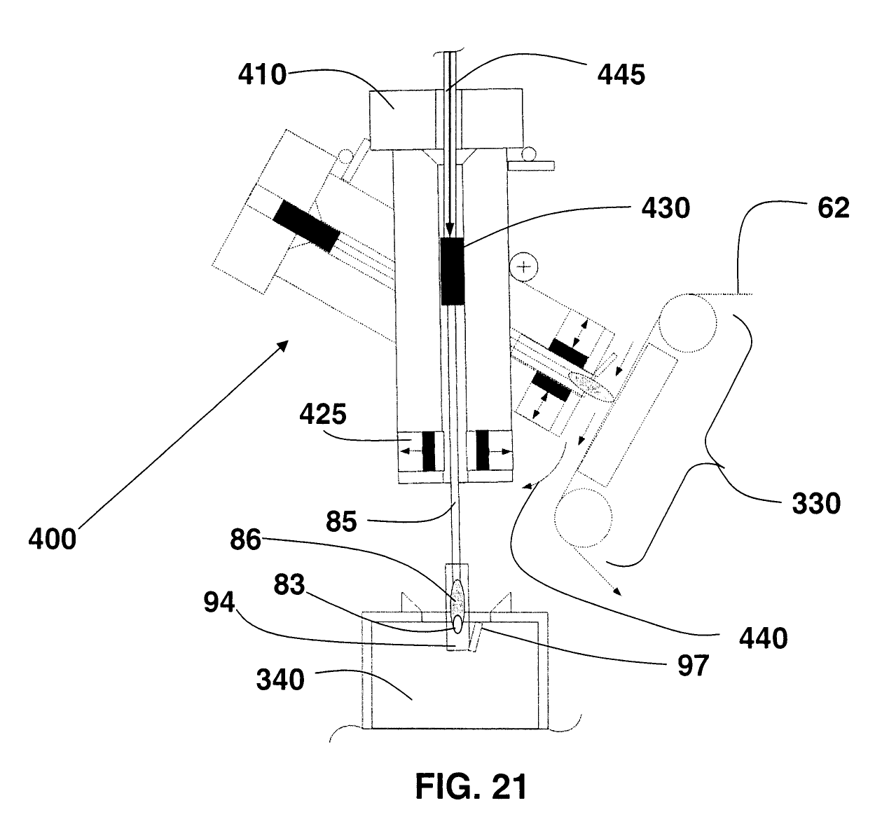

Still referring to FIGS. 18, 19, and 20 the user 305 (shown in FIG. 17A) places the swab assembly 84 into the slot 405 as indicated by the arrow 415. The user then closes the plunger assembly 410 as indicated by arrow 420 causing a gripper mechanism 425 to grip the protective enclosure 94 while the plunger 430 forces the swab 85 to open the enclosure cap 97 contacting the swab sampling tip 86 and the sample 83 to the sampling area 65′ on the web 62. The web support assembly 330 moves the web 62 in the direction indicated by the arrows 435 applying the sample 83 to the sampling area 65′. Now referring to FIG. 21, the transfer apparatus 400 rotates in the direction indicated by arrow 440, positioning the transfer apparatus over the medical waste disposal unit 340. The plunger 430 then ejects the swab assembly 84 directly into the medical waste disposal unit 340 as indicted by arrow 445. The transfer assembly 400 then rotates back to its original position shown in FIG. 18 with the plunger assembly in the open position.

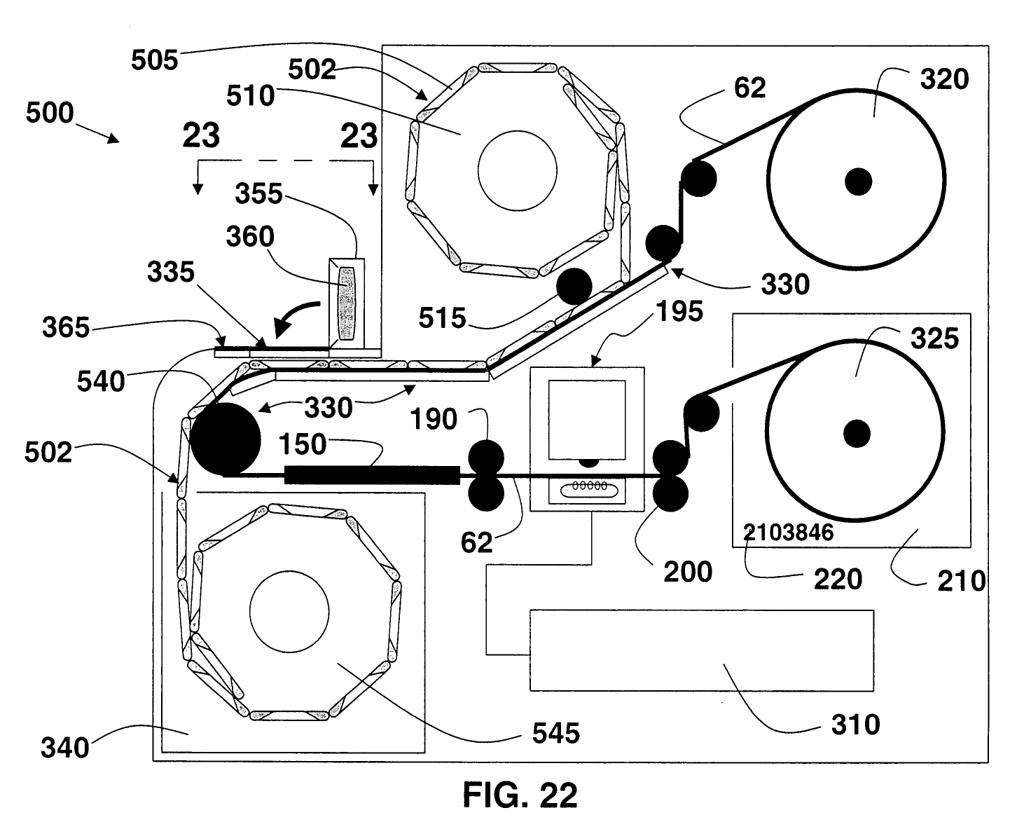

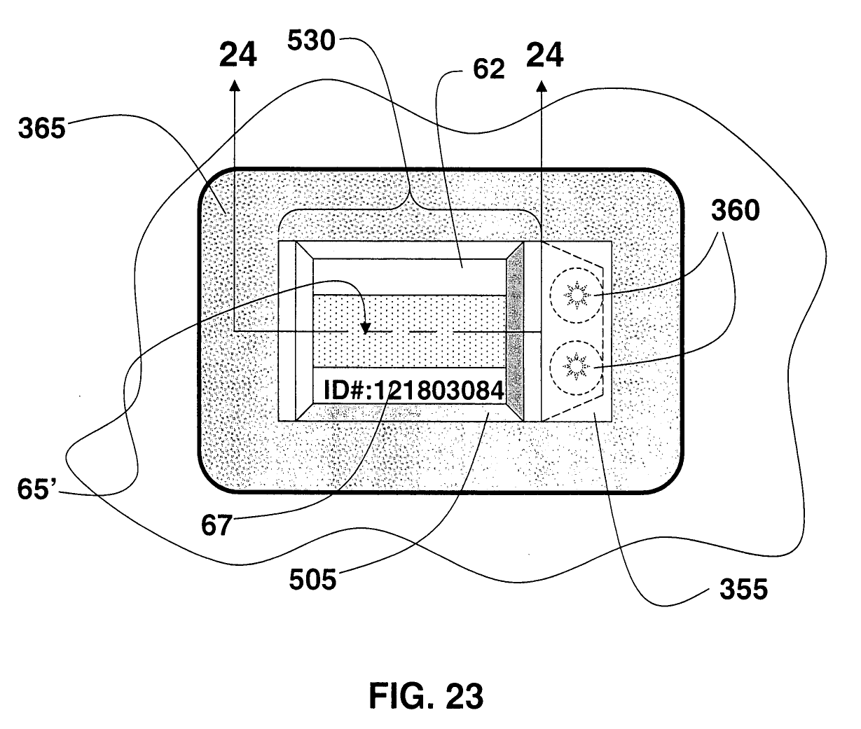

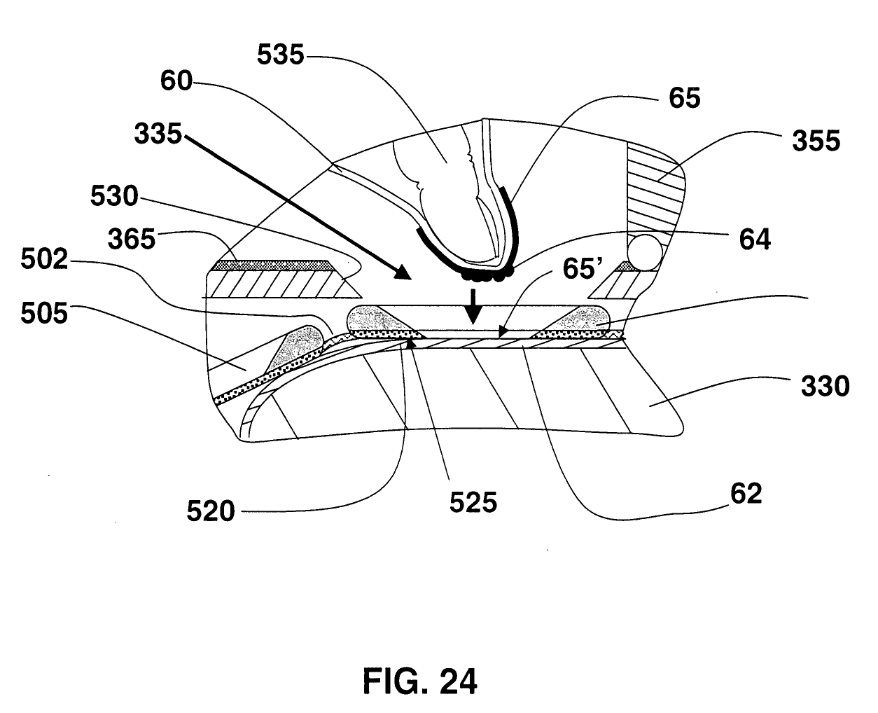

Referring to FIG. 22 there is illustrated another embodiment of an apparatus 500 for applying the sample 64 to the test web 62 in the kiosk's sample placement area 335 made in accordance with the present invention, like parts indicating like parts and operation as previously described. In the embodiment shown, the apparatus 500 is used to apply the sample 64 from the sampling area 65 of the test strip 60 or a swab 85 (not shown) to the sampling area 65′ on the web 62 in a kiosk 300 (shown in FIG. 17A) without causing cross-contamination or contaminating the kiosk sample placement area 335. A removable anti-microbial sheet 365 is used to protect the surfaces around the sample placement area 335. The anti-microbial sheet is described in copending and commonly-assigned U.S. Patent Application Publication No. 2005/0129937 (Patton et al.), incorporated herein by reference. To prevent cross-contamination a continuous web 502 of disposable shields 505 is fed from a supply reel 510 to the web transport assembly 330 where the web 502 of disposable shields 505 is mated to the test web 62 via a pressure roller 515. A low tack adhesive 520 such as used on Post-It Notes® on the bottom side 525 of the disposable shield 505 as shown in FIG. 24 temporarily adheres the disposable shield 505 to the test web 62 as the two webs are transported via the web transport assembly 330 to the sampling area 335. The web transport assembly 330 under the control of the microprocessor 310 moves the disposable shield 505 and test web 62 into position in the sampling area 335 aligning the shield 505 with the sampling area aperture 530 as shown in FIG. 23.

Now referring to FIG. 24, the user is shown using their finger 535 to transfer the sample 64 from the sampling area 65 of the test strip 60 to the sampling area 65′ of the test web 62 in the kiosk.

Referring back to FIG. 22, the user 305 then closes the sample door 355 causing the microprocessor 310 to transport the web 502 of disposable shields and the sampling web 62 over a separation roller 540 where the two webs are separated. The sampling web 62 then passes through the heat processor 150 and scanner 195 where the test web 62 is processed and electronically scanned using any of the methods previously described. The separated web 502 of disposable shields 505 is transported into the medical waste disposal unit 340 where the web 502 is taken up by the take up reel 545.

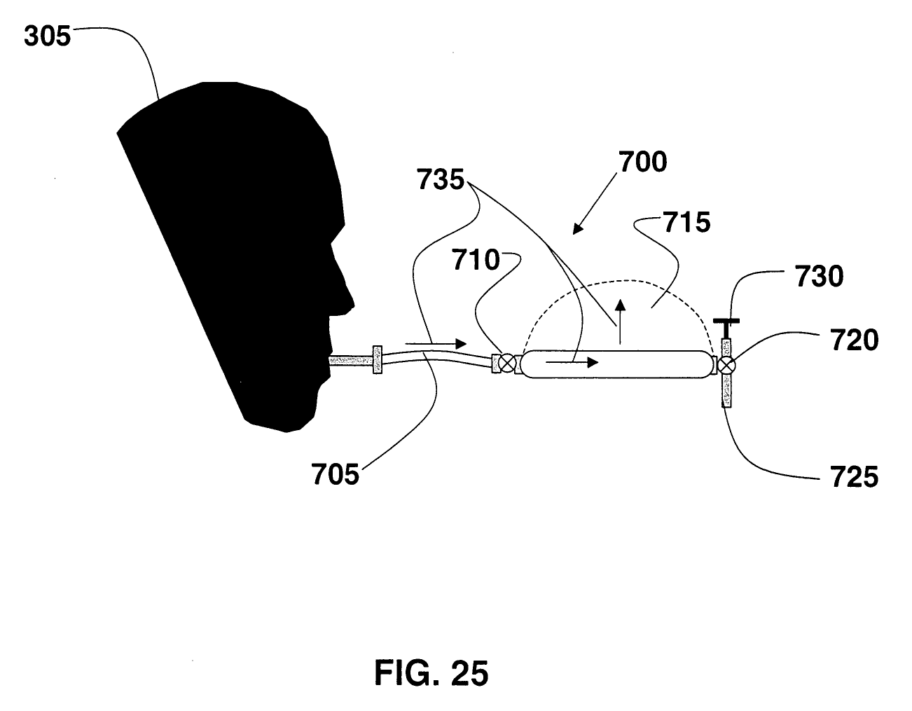

Referring now to FIG. 25, the user 305 is shown breathing into a breath capture kit 700 for obtaining a breath sample from the user 305. The breath capture kit 700 comprised an entry tube 705, a one-way valve 710, an expandable bladder 715, a one-way exit value 720, an exit tube 725, and a release button 730.

The user 305 exhales into the breath capture kit 700 filling the expandable bladder 715. The bladder 715 is design to insure the user 305 must take deep breaths and exhale completely to file the bladder 715. The expelled breath enters and fills the bladder 715 via the entry tube 705 and one-way valve 710 as indicated by the arrows 735.

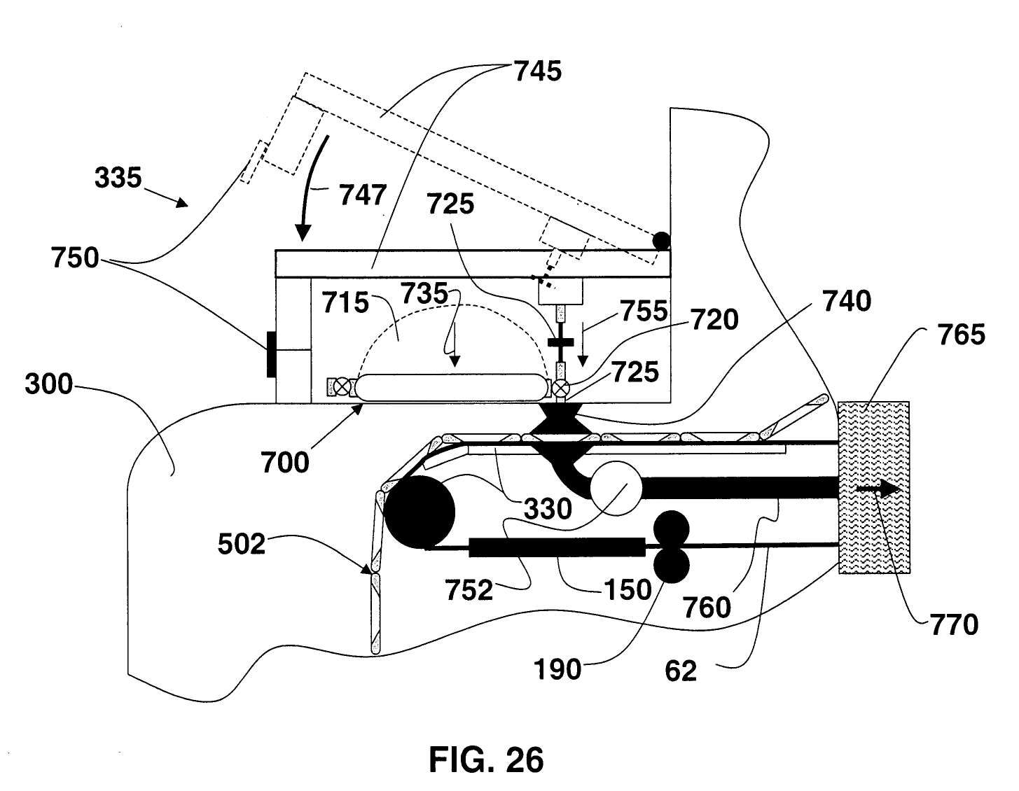

Referring to FIG. 26 there is illustrated another embodiment of an apparatus 500 for applying the breath sample in the breath capture kit 700 to the test web 62 in the kiosk's sample placement area 335 made in accordance with the present invention, like parts indicating like parts and operation as previously described. In the embodiment shown, the user 305 plugs the exit tube 725 of the breath capture kit 700 into a coupling 740 located on the sample placement area 335 and closes the door 745 of the sample placement area 335. As the door 745 closes, indicated by arrow 747, the door 745 latches via door latch 750 and depresses the release button 730. A vacuum pump 752 draws the breath sample from the bladder 715 through one-way value 720 and out the exit tube 725 via the coupling 740 as indicated by arrow 755. The breath sample is draw over and around the web 62 and out of the kiosk 300 via exhaust tube 760. The sample is then exhaust through a filter 765 containing the AgX anti-microbial material as previously described. The air of the breath sample as it passed through the filter 765, as indicated by arrow 770, is cleansed of pathogens by the AgX anti-microbial material. The filter 765 may be for example a Hepa^{R} Filter, which may be used to further filter out the pathogens from the exhausted air. In another embodiment of the present invention, the breath sample is ducted into a gas chromatograph mass spectrometer where the sample is analyzed pathogens.

The invention has been described in detail with particular reference to certain preferred embodiments thereof, but it will be understood that variations and modifications can be effected within the scope of the invention.

### Parts List

5 multilayer sensor
10 support layer
15 signal amplification layer
18 top surface
20 sampling layer
22 top surface
25 light-blocking layer
30 top surface
35 removable protective layer
37 anti-microbial layer
40 subbing layer
45 release layer
50 arrow
55 top surface
57 blocking layer
58 bottom surface
59 test kit
60 test strip
61 sampling patch
62 web
63 non-sampling area
64 sample
65 sampling area (test strip)
65′ sampling area (test web)
65″ sampling area (test strip array)
67 printed data (unique ID #)
68 writeable area
69 test strip array
70 arrow
72 suspect area
75 test area
77 skin
80 arrow
81 arrow
82 arrow
83 sample
84 swab assembly
85 swab
86 swab sampling tip
87 arrow
90 arrow
93 arrow
94 protective enclosure
97 enclosure cap
100 heat processor
105 entry port
110 entry drive rollers
115 heating platens
120 exit port
125 exit drive rollers
140 arrow
145 UV light source
150 heat processor
155 entry port
160 heated rollers
165 exit port
170 apparatus
175 entry slot
176 entry slot door
177 entry slot door
180 entry drive rollers
185 exit port
190 exit drive rollers
195 scanner
200 scanner drive rollers
205 microprocessor
210 disposal box
215 entry port
220 unique identification number
250a test area
250b test area
250c test area
250d test area
255 darkened sampling area
260 unchanged sampling area
265 bottom surface
268 home
270 home diagnostic unit
272 computer
274 Internet
276 scanner
278 monitor
280 printed results and instructions
282 printer
300 kiosk
305 user
310 microprocessor
315 test kit dispensing assembly
320 supply roll
325 take up roll
330 web transport assembly
335 sample placement area
340 medical waste disposal unit
345 printing unit
347 card port
350 monitor
355 sampling area door
360 UV light source
365 anti-microbial sheet
370 fan
372 arrow
375 filter
380 communications network
385 server
391 server
392 server
393 server
400 transfer apparatus
405 slot
410 plunger assembly
415 arrow
420 arrow
425 gripper mechanism
430 plunger
435 arrow
440 arrow
445 arrow
500 apparatus
502 web
505 disposable shields
510 supply reel
515 pressure roller
520 low tack adhesive
525 bottom side
530 aperture
535 finger
540 separation roller
545 take up reel
600 test complete step
605 diagnosis received step
610 question results destination step
615 results sent step
620 question appointment step
625 doctor and time chosen step
630 question make co-pay step
635 co-pay made step
640 transaction and test completed step
700 breath capture kit
705 entry tube
710 one way valve
715 expandable bladder
720 one way exit value
725 exit tube
730 release button
735 arrow
740 coupling
745 door
747 arrow
750 door latch
752 vacuum pump
755 arrow
760 exhaust tube
765 filter
770 arrow

What is claimed is:

1. A remote diagnostic device for medical testing comprising:
a kit dispenser;
a sampling device contained in said kit;
a receiving web for receiving a sample after use by a user;
wherein said receiving web forms a latent image of any pathogens present in said sample;
a processor for developing said latent image;
a scanner for detecting a developed image; and
a microprocessor for analyzing said scanned image for pathogens.

2. A diagnostic device as in claim 1 wherein said sampling device is a breath capture kit.

3. A diagnostic device as in claim 2 wherein breath exhausted from said breath capture kit subsequently is passed through a filter impregnated with an AgX antimicrobial material, compound or composition.

4. A diagnostic device as in claim 1 wherein air exhausted from said diagnostic device is passed through a filter impregnated with an AgX antimicrobial material, compound or composition.

5. A diagnostic device as in claim 1 wherein said diagnostic device is a kiosk.

6. A diagnostic device as in claim 1 wherein said diagnostic device accepts credit cards or medical cards or both.

7. A diagnostic device as in claim 1 wherein said diagnostic device is connected to a remote server.

8. A diagnostic device as in claim 7 wherein said remote server recommends appointments with a doctor or a medical specialist based on said analysis of said scanned images.

9. A diagnostic device as in claim 8 wherein said diagnostic device offers said user an opportunity to pay a medical co-pay.

10. A diagnostic device as in claim 8 wherein said diagnostic device provides a referral to said medical specialist for said user's appointment.

11. The diagnostic device of claim 1 wherein the sampling device is a swab for removing said sample from said user.

12. The diagnostic device of claim 1 wherein a removable anti-microbial disposable sheet protects surfaces around a sample receiving area.

13. The diagnostic device of claim 1 wherein a removable disposable shield protects said receiving web.

14. A remote diagnostic device for medical testing comprising:
a kit dispenser;
a sampling device contained in said kit;
a receiver for receiving a sample after use of said sampling device by a user;
wherein said sample forms a latent image of any pathogens present in said sample;
a processor for developing said latent image;
a scanner for detecting a developed image; and
a microprocessor for analyzing said scanned image for pathogens.

15. The remote diagnostic device of claim 14 wherein the sampling device is a test strip having a sample area for receiving said sample.

16. The remote diagnostic device of claim 15 wherein said test strip sampling area is surrounded by a non-sampling area comprising an anti-microbial substance.

17. The remote diagnostic device of claim 16 wherein said anti-microbial substance is silver based compound.

18. A method for remotely administering a medical test comprising:
dispensing a test kit containing a sampling device;
applying said sampling device to a user;
depositing said sampling device on a receiving web;
forming a latent image of any pathogens present in said sampling device;
developing said latent image;
scanning said developed image; and
analyzing said scanned image for pathogens.

19. The test strip of claim 1 wherein the sampling area is surrounded by the non-sampling area.

20. A remote diagnostic device for medical testing comprising:
a sampling device;
a receiver for receiving a sample after use of said sampling device by a user;
wherein said sample forms a latent image of any pathogens present in said sample; and
a processor for developing said latent image.

21. A remote diagnostic device as in claim 20 wherein a scanner creates an image file of said developed image

22. A remote diagnostic device as in claim 21 wherein a microprocessor analyses said scanned image for pathogens.

23. A remote diagnostic device as in claim 22 wherein results of said analysis is transmitted to a doctor.

24. A remote diagnostic device as in claim 23 wherein said doctor transmits a prescription or instructions to said microprocessor.

25. A remote diagnostic device as in claim 24 wherein said prescriptions or instructions are printed or displayed.

26. A remote diagnostic device as in claim 21 wherein a microprocessor analyses said scanned image for pathogens and wherein said microprocessor is remotely located from said diagnostic device.
